(19) United States
(12) Patent Application Publication
(10) Pub. No.: US 2025/0027113 A1
(43) Pub. Date: Jan. 23, 2025

(54) METHODS OF INTRODUCING A FOREIGN SUBSTANCE INTO A CELL

(71) Applicants: WaveCyte Biotechnologies LLC, Plymouth, MN (US); Royal Melbourne Institute of Technology, Melbourne (AU)

(72) Inventors: Steve Wanjara, Plymouth, MN (US); Elena Ivanova, Melbourne (AU); The Hong Phong Nguyen, Melbourne (AU); Tharushi Perera, Melbourne (AU); Rodney Croft, Melbourne (AU); Denver Linklater, Melbourne (AU); Saulius Joudkazis, Melbourne (AU)

(73) Assignees: WaveCyte Biotechnologies LLC, Plymouth, MN (US); Royal Melbourne Institute of Technology, Melbourne (AU)

(21) Appl. No.: 18/774,322

(22) Filed: Jul. 16, 2024

Related U.S. Application Data
(60) Provisional application No. 63/514,007, filed on Jul. 17, 2023.

(51) Int. Cl. C12N 15/87

(52) U.S. Cl. CPC C12N 15/87; C12N 2529/00

(57) ABSTRACT

Provided herein are methods for introducing a foreign substance into a cell using high frequency electromagnetic energy. Provided are methods for delivery substances to both prokaryotic and to eukaryotic cells. Also provided are cells generated with the methods disclosed herein.

### FIELD OF THE INVENTION

The present disclosure relates to methods for introducing a foreign substance into a cell using high frequency electromagnetic energy.

### BACKGROUND

Intracellular delivery of foreign substances such as foreign nucleic acids into eukaryotic cells (referred to as transfection) or prokaryotic cells (referred to as transformation) to modify the host cell's genome has become an indispensable technique in the fields of biomedical engineering, synthetic biology, hematology, immunology and oncology. However, efficient and safe transfection of nucleic acids into cells remains a key bottle neck in cell engineering.

Existing non-viral technologies deployed for intracellular delivery of molecules utilize techniques such as strong electrical fields (electroporation), nanoparticles, sonoporation, or pore-forming chemicals and microinjection. However, these methods come with numerous complications such as significant loss of cell viability, low transfection efficiencies, non-specific molecule delivery, modification or damage to the payload molecules, low throughput, and/or difficult implementation. Moreover, these intracellular delivery methods often result in ineffective delivery of molecules to cells with cell walls, such as prokaryotic, algal, yeast, and plant cells. Thus, there is an unmet need for techniques for delivering foreign substances into cells, particularly for techniques that are highly effective in delivering a range of molecules to a variety of cell types. In addition, techniques that allow for automation, rapid, high throughput intracellular delivery would improve intracellular delivery of foreign substances such as nucleic acids and allow for larger scale clinical, manufacturing, and drug screening applications.

### SUMMARY

Provided herein are methods for achieving intracellular delivery of nucleic acids into host cells using high frequency electromagnetic energy (HF EME).

In one aspect, provided is a method for introducing a foreign substance into a cell, the method comprising: (a) providing a plurality of cells suspended in a first solution; (b) exposing the plurality of cells to an electromagnetic field with a frequency of about 6 to about 35 GHZ; (c) allowing the temperature in the first solution to decrease; (d) repeating steps (b) and (c) one or more times; and (c) contacting the plurality of cells with the foreign substance, wherein the temperature of the first solution in steps (b) to (d) does not exceed about 37° C. In some embodiments, steps (b) and (c) are repeated two, three, four, five, or six times prior to step (e). In some embodiments, steps (b) and (c) are repeated twice prior to step (e). In some embodiments, the plurality of cells is exposed to the electromagnetic field for about 30 to about 120 s in step (b). In some embodiments, the plurality of cells is exposed to the electromagnetic field for about 40 s to about 100 s in step (b). In some embodiments, the plurality of cells is exposed to the electromagnetic field for about 40 s to about 65 s in step (b). In one embodiment, the plurality of cells is exposed to the electromagnetic field for about 30 s in step (b). In one embodiment, the plurality of cells is exposed to the electromagnetic field for about 45 s in step (b). In one embodiment, the plurality of cells is exposed to the electromagnetic field for about 60 s in step (b). In some embodiments, in step (c), the temperature in the first solution is allowed to decrease for about 30 s to about 300 s. In some embodiments, in step (c), the temperature in the first solution is allowed to decrease for about 60 s to about 200 s. In one embodiment, in step (c), the temperature in the first solution is allowed to decrease for about 120 s. In one embodiment, the plurality of cells is exposed to the electromagnetic field for about 45 s in step (b), the temperature in the first solution is allowed to decrease for about 120 s in step (c), and steps (b) and (c) are repeated two times. In one embodiment, the plurality of cells is exposed to the electromagnetic field for about 60 s in step (b), the temperature in the first solution is allowed to decrease for about 120 s in step (c), and wherein steps (b) and (c) are repeated two times. In one embodiment, the plurality of cells is exposed to the electromagnetic field for about 30 s in step (b), the temperature in the first solution is allowed to decrease for about 120 s in step (c), and wherein steps (b) and (c) are repeated five times. In one embodiment, in step (c), the temperature in the first solution is allowed to decrease to about 30° C. In one embodiment, in step (c), the temperature in the first solution is allowed to decrease to about 27° C. In one embodiment, in step (c), the temperature in the first solution is allowed to decrease to about 25° C. In one embodiment, in step (c), the temperature in the first solution is allowed to decrease to about 24° C. In some embodiments, the method further comprising a step of substantially purifying the plurality of cells after step (d) and before step (e).

In one aspect, provided is a method for introducing a foreign substance into a cell, the method comprising (a) providing a plurality of cells suspended in a first solution; (b) exposing the plurality of cells to an electromagnetic field with a frequency of about 6 to about 35 GHz; and (c) contacting the plurality of cells with the foreign substance, wherein the temperature of the first solution in step (b) does not exceed about 37° C. In one embodiment, the method is performed in a device comprising a temperature control unit. In some embodiments, step (b) is repeated two, three, four, five, or six times prior to step (c). In some embodiments, the plurality of cells is exposed to the electromagnetic field for about 30 to about 120 s in step (b). In some embodiments, the plurality of cells is exposed to the electromagnetic field for about 40 s to about 100 s in step (b). In some embodiments, the plurality of cells is exposed to the electromagnetic field for about 40 s to about 65 s in step (b). In one embodiment, the plurality of cells is exposed to the electromagnetic field for about 30 s in step (b). In one embodiment, the plurality of cells is exposed to the electromagnetic field for about 45 s in step (b). In one embodiment, the plurality of cells is exposed to the electromagnetic field for about 60 s in step (b). In some embodiments, the method further comprising a step of substantially purifying the plurality of cells after step (b) and before step (c).

In some embodiments, the electromagnetic field has a frequency of about 10 to about 30 GHz. In some embodiments, the electromagnetic field has a frequency of about 16 to about 20 GHz. In one embodiment, the electromagnetic field has a frequency of about 18 GHZ.

In some embodiments, the first solution is a first buffer. In some embodiments, the first solution has a pH of about 6 to about 8.

In one embodiment, the foreign substance is a nucleic acid. In some embodiments, the nucleic acid is a deoxyribonucleic acid (DNA) or ribonucleic acid (RNA). In some embodiments, the nucleic acid is a DNA or RNA comprising one or more modified nucleotides that increase the stability or half-life of the DNA or RNA in vivo or in vitro. In one embodiment, the DNA is methylated DNA. In one embodiment, the nucleic acid is a naturally occurring chromosome or a portion thereof. In one embodiment, the nucleic acid is an expression vector.

In some embodiments, the foreign substance is a polypeptide, sugar, or a small molecule.

In one embodiment, the plurality of cells are substantially purified by centrifugation and replacement of the first solution with a second solution comprising the foreign substance. In one embodiment, the second solution is a second buffer. In some embodiments, the second solution has a pH of about 6 to about 8.

In one embodiment, the foreign substance is a nucleic acid comprising a transgene encoding for a polypeptide. In one embodiment, the method further comprises a step of incubating the plurality of cells under conditions allowing for the expression of the polypeptide after the step of contacting the plurality of cells with the foreign substance. In one embodiment, the nucleic acid comprises a transgene encoding for an RNA. In some embodiments, the RNA is a siRNA, antisense oligonucleotide, or an RNAi. In one embodiment, the method further comprising a step of incubating the plurality of cells under conditions allowing for the production of the RNA after the step of contacting the plurality of cells with the foreign substance. In one embodiment, the step of incubating the plurality of cells is performed at about 37° C.

In one embodiment, the plurality of cells is a plurality of bacterial cells. In one embodiment, the bacterial cells are Escherichia coli cells. In one embodiment, the plurality of cells is a plurality of eukaryotic cells. In some embodiments, the eukaryotic cells are immune cells or immune cell-derived cells. In one embodiment, the eukaryotic cells are primary cells. In one embodiment, the eukaryotic cells are pluripotent cells. In one embodiment, the eukaryotic cells are hematopoietic stem cells. In some embodiments, the eukaryotic cells are Jurkat cells, PC-12 cells, or HeLa cells.

In one aspect, provided is a cell comprising a foreign substance, wherein the cell is generated using a method disclosed herein.

### BRIEF DESCRIPTION OF THE DRAWINGS

FIGS. 1A, 1B. and 1C illustrate the experimental set up for the HF EME-induced genetic transformation of E. coli M 109 bacterial cells. FIG. 1A. Schematic of the high frequency electromagnetic field (HF EMF) chamber of the Vari-Wave Model LT 1500 microwave. A ceramic pedestal for the sample treatment is in the chamber. The bacterial suspension (2 mL) in the micro petri dish was subjected to three discrete 60 s exposures with a cooling period of 2 min between exposures. FIG. 1B. Schematic representation of pGLO plasmid DNA uptake in E. coli exposed to HF EME. The plasmid DNA was added at a concentration of 100 ng/μL. The samples were outgrown in LB broth supplemented with 100 μg/mL ampicillin and assessed for GFP expression. FIG. 1C. Incident power density of the sample being exposed. The top 10% exposure group of the E. coli population received a PD of over 30 kW m⁻² (far right bar), whereas the 10% with lowest exposure were subjected to an averaged PD of just over 5.6 kw m⁻² (far left bar).

FIG. 2 shows GFP expression in E. coli JM 109 following exposure to HF EME after 24 h. pGLO plasmid DNA transformation was visualized using fluorescence microscopy. Green fluorescence from expressed GFP was detected in the HF EME exposed bacterial cells. HF EME: Cells treated with HF EME and incubated with plasmid DNA. HS: Cells exposed to heat shock and incubated with plasmid DNA. Sham (peltier treated control treated with DNA): Cells exposed to same increases in temperature resulting from HF EME treatment and incubated with plasmid DNA. Control: No heat/HF EME treatment, no DNA.

FIG. 3 illustrates GFP expression in E. coli JM 109 following exposure to HF EME after 24 h. Percent GFP expression was determined by flow cytometry 24 h after exposure to HF electromagnetic energy (HF EME), heat shock, sham (pp) or untreated control sample. Experimental conditions as in FIG. 2. Shown is the population distribution of GFP positive cells in the various sample constructs by flow cytometry. Statistical significance is denoted by *p<0.05 and ** p<0.01.

FIGS. 4A and 4B compare different methods for the transformation of bacterial cells. FIG. 4A. Comparison of number of colony forming units (CFU) per μg plasmids DNA for no treatment controls, chemical transformation (heat shock, HS), sham (peltier plate heated cells treated with DNA) and HF EME methods. Experimental conditions as in FIG. 2. Mean values and standard deviations are shown. The transformation efficiency was calculated after 24 h on selective media. Statistical significance is denoted by *p<0.05 and ** p<0.01. FIG. 4B. Relative fold change in transformation efficiency (CFU/μg) for HF EME method as compared to chemical transformation (heat shock) method. Experimental conditions as in FIG. 2.

FIGS. 5A, 5B, 5C, 5D, 5E, 5F, 5G, and 5H show TEM and Cryo-TEM micrographs of the plasmid DNA and bacterial interactions following HF EME. FIG. 5A. Unexposed E. coli cell. FIG. 5B. Interactions of the plasmid DNA with HF EME exposed E. coli; the pool of DNA (arrows) is attached to the bacterial cell membrane. Plasmid DNA has a length of 344.3 nm in its double helical form. FIG. 5C. TEM micrograph of the plasmid DNA. FIG. 5D. The pGLO plasmid exists cither as circular or coiled DNA FIGS. 5E and 5F. Interactions of the plasmid DNA with HF EME exposed E. coli using cryo-TEM FIGS. 5G and 5H. The circular plasmid interacting with the outer membrane of E. coli exposed to HF EME can be visualized clearly (arrow).

FIG. 6 shows confocal laser scanning microscopy (CLSM) analysis of the silica nanospheres in the 18 GHz EME exposed and unexposed Jurkat cells. Typical CLSM images showed fluorescent silica nanospheres being internalized by both the resting and activated Jurkat cells after exposing to 18 GHz EME. A smaller number of resting and activated unexposed control cells also had a fluorescent signal from the silica nanospheres. Scale bars are 10 μm.

FIG. 7 shows transmission electron microscopy analysis of the silica nanospheres in the 18 GHz EME exposed and unexposed Jurkat cells. Typical TEM images showed that both the resting and activated Jurkat cells exposed to 18 GHz EME were able to internalize silica nanospheres as indicated in the black circles. No nanosphere internalization was detected and the nanosphere clusters could be seen sticking to the membrane of the unexposed control group. Scale bars are 2 μm.

FIGS. 8A and 8B show flow cytometry analysis of the silica nanospheres in the 18 GHZ EME exposed and unexposed Jurkat cells. FIG. 8A. The fluorescein-5-isothiocyanate (FITC) fluorescence of silica nanospheres (x-axis) and cells side-scattered light SSC (y-axis) were measured and plotted in the scatterplot graphs. The fraction of cells that had the silica nanospheres (FITC positive) was gated and the percentage of the total population was shown. Median Fluorescence Intensity (MFI) of each sample plotted on a bar chart suggested a good signal to noise ratio and there were actual shifts in the fluorescent intensity of the samples. FIG. 8B. Data are presented as Mean±SD and are representative of three independent repeat experiments. Statistical analysis was performed by using Tukey-Kramer procedure for one-way ANOVA analysis. Statistically significant differences for each silica uptake level between EME exposed and unexposed cells were indicated on top of the corresponding graphic bars (ns P>0.05, *P<0.05).

FIGS. 9A, 9B, and 9C show GPF expression in 18 GHz EME exposed and unexposed Jurkat cells. FIG. 9A. CLSM analysis of the expressed GFP in the 18 GHz EME exposed and unexposed Jurkat cells. Typical CLSM images showed GFP being expressed by both the resting and activated Jurkat cells after exposing to 18 GHz EME and mRNA Lipofectamine complex. A smaller number of resting control cells also expressed GFP due to the Lipofectamine. However, none of the activated unexposed control cells was fluorescent. Scale bars are 5 μm. FIG. 9B. Flow cytometry analysis of the expressed GFP in the 18 GHz EME exposed and unexposed Jurkat cells. The GFP (x-axis) and cells side-scattered light SSC (y-axis) were measured and plotted in the scatterplot graphs. The fraction of cells that successfully transfected with mRNA Lipofectamine complex (GFP positive) was gated and the percentage of the total population was shown. MFI of each sample plotted on a bar chart suggested a good signal to noise ratio and there were actual shifts in the fluorescent intensity of the samples. FIG. 9C. Data are presented as Mean±SD and are representative of three independent repeat experiments. Statistical analysis was performed by using Tukey-Kramer procedure for one-way ANOVA analysis. Statistically significant differences for each GFP expression level between EME exposed and unexposed cells were indicated on top of the corresponding graphic bars (ns P>0.05, *P<0.05).

FIG. 10 shows quantification of viable Jurkat cells after 18 GHz EME exposure. The number of viable cells in the EME exposed and the unexposed control groups varied slightly; no significant changes (P>0.05) were detected. Data are presented as Mean±SD and are representative of three independent repeat experiments. Statistical analysis was performed by using Tukey-Kramer procedure for one-way ANOVA analysis. Statistically significant differences for each viable cell number between EME exposed and unexposed cells were indicated on top of the corresponding graphic bars (ns P>0.05, *P<0.05).

FIGS. 11A and 11B show transfection of PC-12 cells with dasher GFP mRNA or a plasmid expressing GFP. FIG. 11A. Transfection of PC-12 cells with dasher GFP mRNA. Transfection of PC-12 cells post EME exposure was compared to control cells not exposed to EME but had dasher eGFP mRNA added. PC-12 cells were imaged 24 h or 48 h later to detect the expression of eGFP. A green signal was detected 24 h later, the intensity of the signal appears to be very low. 48 h post treatment showed stronger eGFP signal in EME treated cells. Controls did not emit any green signal/eGFP signal. FIG. 11B. Transfection of PC-12 cells with plasmid DNA encoding for green fluorescent protein (GFP). Transfection of PC-12 cells post EME exposure was determined using confocal laser scan microscopy to compare PC-12 cells exposed to or not exposed to HF EME followed by addition of plasmid DNA encoding GFP protein. The PC-12 cells were imaged 48 h later to detect the expression of eGFP.

### DETAILED DESCRIPTION

Methods of Introducing a Foreign Substance into a Cell

Provided herein are methods for introducing a foreign substance into a cell using a high frequency electromagnetic energy (HF EME). As used herein, the term “high frequency electromagnetic energy” refers to an electromagnetic field in the frequency range between 100 kilohertz (kHz) and 300 gigahertz (GHz). The foreign substance may be a compound or a composition not previously present in the cell. Examples of a foreign substance include, but are not limited to, a particle, a small molecule, a nucleic acid, a polypeptide, a lipid, or a sugar.

Provided herein are methods that are useful for bacterial transformation. As used herein, bacterial transformation refers the uptake of extracellular DNA from the environment into the cell cytoplasm. Exogenous DNA can usually only be internalized by bacterial cells that have previously developed genetic competence, cither naturally or artificially. Natural bacterial transformation was first discovered in 1928 in Gram-positive Streptococcus pneumoniae bacterial cells. Internalization of exogenous DNA and integration into the recipient genome via homologous recombination results in bacteria acquiring new genetic traits and enables adaption to environmental changes. In modern times, genetically engineered bacteria are an industry standard for many applications including drug manufacturing, production of enzymes for food, nanomaterial (nanoparticle) synthesis, and environmental bioremediation. Electroporation is a commonly used bacterial transformation technique since it allows for the uptake of very large sequences of genetic material, e.g., large sized plasmids and bacterial artificial chromosomes (BACs in the range of 150-350 kb). The cell membrane is usually permeabilized using electrical pulses at high voltages (1000-3000 V) that increase the transmembrane potential (TMP) above 0.2 V. However, electroporation can cause the formation of irreversible pores in cells due to the harsh treatment conditions, resulting in cell lysis and death and low transformation efficiencies in a laboratory setting. Another laboratory standard for bacterial transformation, heat shock, requires chemically competent cells and is generally less efficient than electroporation, requiring larger amounts of extracellular DNA. Likewise, the limitations of sonoporation are low efficiency and damage to the target cells, whereas microinjection techniques can only be applied to a limited number of target cells. Hence, there is ongoing demand for the development of more efficient genetic material delivery methods that ensure the safe introduction of genetic material into the cell without compromise of the host cell viability.

Also provided herein are methods that are useful for transfection. As used herein, transfection refers to the delivery of nucleic acids into a eukaryotic cell. Transfection, which allows the modification of a cell's genome, is a crucial technique for gene therapy, vaccine development, and molecular research due to the technique's ability to interfere with cellular processes and molecular mechanisms of diseases. Problematically, some cell types, including primary and stem cells, are notoriously difficult to transfect using current transfection methods. In addition, transfecting cells in suspension is generally considered more difficult than transfecting adherent cells because the transfection complex of suspension cells has a lower chance of attaching to the cell surface. For example, the Jurkat cell line, a human T-cell leukemia cell line, is a well-known type of cell that is challenging to transfect. Jurkat cells have been widely used as a model system for T cell signaling and activation due to their uniform genetic background and rapid growth rate. However, the high resistance of Jurkat cells to transfection limits their use in gene therapy and basic research applications.

In the methods disclosed herein, a HF EME source may be positioned with respect to a reservoir containing a plurality of cells to be transfected in a cell compatible solution in a microwave cavity chamber/reservoir. A high-power solid-state microwave generator may be activated to produce a high frequency electromagnetic field (HF EMF) in the microwave cavity chamber in a manner effective to enable delivery of a foreign substance to the plurality of cells. The treated cells may subsequently cultured, for example, to allow for the expression of a polypeptide of interest in instances where the foreign substance delivered to the cell is a nucleic acid encoding for said polypeptide. The methods of the instant disclosure enable the intracellular delivery of molecules into target cells with minimal cell death as compared to traditional mechanical cell transformation or transfection methods such as electroporation, resulting in a higher number of cells that have taken up the foreign substance. A further benefit of the HF EMF/EME transfection method disclosed herein is that it can fully be automated, thereby further reducing sample handling and minimizing exposure of a population of cells to blood borne pathogens such as viruses or parasites. Further, the amount of sample to be processed can be scaled up with minimal optimization.

In one aspect, provided is a method of introducing a foreign substance into a cell, the method comprising: (a) providing a plurality of cells suspended in a first solution; (b) exposing the plurality of cells to an electromagnetic field with a frequency of about 6-35 GHZ; (c) allowing the temperature in the first solution to decrease; (d) repeating steps (b) and (c) one or more times; and (c) contacting the plurality of cells with the foreign substance. In one embodiment, the temperature of the first solution in steps (b) to (d) does not exceed about 39° C. In one embodiment, the temperature of the first solution in steps (b) to (d) does not exceed about 38° C. In one embodiment, the temperature of the first solution in steps (b) to (d) does not exceed about 37° C. In one embodiment, the temperature of the first solution in steps (b) to (d) does not exceed about 36° C. In one embodiment, the temperature of the first solution in steps (b) to (d) does not exceed about 35° C. In one aspect, provided is a method of introducing a foreign substance into a cell, the method comprising: (a) providing a plurality of cells suspended in a first solution; (b) exposing the plurality of cells to an electromagnetic field with a frequency of about 6-35 GHZ; (c) allowing the temperature in the first solution to decrease; (d) repeating steps (b) and (c) one or more times; and (c) contacting the plurality of cells with the foreign substance, wherein the temperature of the first solution in steps (b) to (d) does not exceed about 37° C. In embodiments, steps (b) and (c) (i.e., the exposing and the allowing the temperature in the first solution to decrease steps) are repeated one, two, three, four, five, six, seven, eight, nine, or ten times before the step of contacting the plurality of cells with the foreign substance. In one embodiment, the steps are repeated twice (i.e., steps (b) and (c) are each performed a total of three times). In embodiments, in step (c), the temperature in the first solution is allowed to decrease to about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., or about 4° C. In one embodiment, in step (c), the temperature in the first solution is allowed to decrease to about 30° C. In one embodiment, in step (c), the temperature in the first solution is allowed to decrease to about 27° C. In one embodiment, in step (c), the temperature in the first solution is allowed to decrease to about 25° C. In one embodiment, in step (c), the temperature in the first solution is allowed to decrease to about 24° C. In one embodiment, in step (c), the temperature in the first solution is allowed to decrease to room temperature.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Other meanings of “about” may be apparent from the context, such as rounding off, so, for example “about 1” may also mean from 0.5 to 1.4. Alternatively, the term “about” refers to within an acceptable standard error of the mean, when considered by one of ordinary skill in the art.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

In one aspect, provided is a method of introducing a foreign substance into a cell, the method comprising providing a plurality of cells suspended in a first solution; exposing the plurality of cells to an electromagnetic field with a frequency of about 6-35 GHz; and contacting the plurality of cells with the foreign substance. In one embodiment, the temperature of the first solution in the exposing step does not exceed about 39° C. In one embodiment, the temperature of the first solution in the exposing step does not exceed about 38° C. In one embodiment, the temperature of the first solution in the exposing step does not exceed about 37° C. In one embodiment, the temperature of the first solution in the exposing step does not exceed about 36° C. In one embodiment, the temperature of the first solution in the exposing step does not exceed about 35° C. In one aspect, provided is a method of introducing a foreign substance into a cell, the method comprising providing a plurality of cells suspended in a first solution; exposing the plurality of cells to an electromagnetic field with a frequency of about 6-35 GHZ; and contacting the plurality of cells with the foreign substance, wherein the temperature of the first solution in the exposing step does not exceed about 37° C.

In one aspect, the plurality of cells is exposed to an electromagnetic field with a frequency of about 6 to about 35 GHz. In some embodiments, the plurality of cells is exposed to an electromagnetic field with a frequency of about 10 to about 30 GHz. In some embodiments, the plurality of cells is exposed to an electromagnetic field with a frequency of about 16 to about 20 GHz. In some embodiments, the plurality of cells is exposed to an electromagnetic field with a frequency of about 6 GHz, about 7 GHZ, about 8 GHZ, about 9 GHz, about 10 GHZ, about 11 GHZ, about 12 GHZ, about 13 GHZ, about 14 GHZ, about 15 GHz, about 16 GHZ, about 17 GHZ, about 18 GHz, about 19 GHz, about 20 GHz, about 21 GHz, about 22 GHZ, about 23 GHZ, about 24 GHz, about 25 GHZ, about 26 GHZ, about 27 GHZ, about 28 GHZ, about 29 GHz, about 30 GHZ, about 31 GHZ, about 32 GHZ, about 33 GHZ, about 34 GHz, or about 35 GHz. In some embodiments, the plurality of cells is exposed to an electromagnetic field with a frequency of at least about 6 GHz, at least about 7 GHZ, at least about 8 GHZ, at least about 9 GHZ, at least about 10 GHz, at least about 11 GHz, at least about 12 GHZ, at least about 13 GHZ, at least about 14 GHz, at least about 15 GHZ, at least about 16 GHZ, at least about 17 GHZ, at least about 18 GHZ, at least about 19 GHz, at least about 20 GHz, at least about 21 GHz, at least about 22 GHz, at least about 23 GHZ, at least about 24 GHZ, or at least about 25 GHz. In some embodiments, the plurality of cells is exposed to an electromagnetic field with a frequency of no more than about 10 GHZ, no more than about 11 GHz, no more than about 12 GHZ, no more than about 13 GHZ, no more than about 14 GHz, no more than about 15 GHZ, no more than about 16 GHZ, no more than about 17 GHz, no more than about 18 GHz, no more than about 19 GHz, no more than about 20 GHz, no more than about 21 GHz, no more than about 22 GHz, no more than about 23 GHZ, no more than about 24 GHz, no more than about 25 GHZ, no more than about 26 GHZ, no more than about 27 GHz, no more than about 28 GHz, no more than about 29 GHz, no more than about 30 GHZ, no more than about 31 GHz, no more than about 32 GHZ, no more than about 33 GHZ, no more than about 34 GHz, or no more than about 35 GHz. In one embodiment, the plurality of cells is exposed to an electromagnetic field with a frequency of about 18 GHz. If the population of cells is exposed to the electromagnetic field more than once, the frequency of the electromagnetic field during the first exposure may or may not be different than the frequency of the electromagnetic field during the one or more subsequent exposures.

In one aspect, the plurality of cells is exposed to the electromagnetic field for about 10 s to about 300 s. As noted herein, the plurality of cells may be exposed to the electromagnetic field more than once. As such, as used herein, the referenced time periods for exposure refer to the exposure during a given step (wherein the exposure step may be repeated more than once). As a non-limiting example, the plurality of cells might be exposed to an electromagnetic field twice for 30 s, for a total exposure time of 60 s. Further, a person skilled to the art may contemplate making certain changes to the exposure period. As a non-limiting example, a person skilled in the art may modify a 30 s exposure period and introduce one, two, or three breaks during the exposure (e.g., 10 s exposure, 1 s break, 10 s exposure, Is break, 10 s exposure). Such variants are encompassed by the instant disclosure. In some embodiments, the plurality of cells is exposed to the electromagnetic field for about 20 s to about 200 s. In some embodiments, the plurality of cells is exposed to the electromagnetic field for about 30 s to about 120 s. In some embodiments, the plurality of cells is exposed to the electromagnetic field for about 40 s to about 100 s. In some embodiments, the plurality of cells is exposed to the electromagnetic field for about 40 s to about 65 s. In some embodiments, the plurality of cells is exposed to the electromagnetic field for about 10 s, about 15 s, about 20 s, about 25 s, about 30 s, about 35 s, about 40 s, about 45 s, about 50 s, about 55 s, about 60 s, about 65 s, about 70 s, about 75 s, about 80 s, about 85 s, about 90 s, about 100 s, about 110 s, about 120 s, about 130 s, about 140 s, about 150 s, about 160 s, about 170 s, about or 180 s. In some embodiments, the plurality of cells is exposed to the electromagnetic field for at least about 10 s, at least about 15 s, at least about 20 s, at least about 25 s, at least about 30 s, at least about 35 s, at least about 40 s, at least about 45 s, at least about 50 s, at least about 55 s, at least about 60 s, at least about 65 s, at least about 70 s, at least about 75 s, at least about 80 s, at least about 85 s, at least about 90 s, at least about 100 s, at least about 110 s, at least about 120 s, at least about 130 s, at least about 140 s, at least about 150 s, at least about 160 s, at least about 170 s, at least about or 180 s. In some embodiments, the plurality of cells is exposed to the electromagnetic field (in a single step) for no more than about 10 s, no more than about 15 s, no more than about 20 s, no more than about 25 s, no more than about 30 s, no more than about 35 s, no more than about 40 s, no more than about 45 s, no more than about 50 s, no more than about 55 s, no more than about 60 s, no more than about 65 s, no more than about 70 s, no more than about 75 s, no more than about 80 s, no more than about 85 s, no more than about 90 s, no more than about 100 s, no more than about 110 s, no more than about 120 s, no more than about 130 s, no more than about 140 s, no more than about 150 s, no more than about 160 s, no more than about 170 s, no more than about or 180 s. In one embodiment, the plurality of cells is exposed to the electromagnetic field for about 30 s. In one embodiment, the plurality of cells is exposed to the electromagnetic field for about 45 s. In one embodiment, the plurality of cells is exposed to the electromagnetic field for about 60 s.

In one aspect, the plurality of cells suspended in solution. In embodiments, the solution has a pH of about pH 5 to about pH 9. In embodiments, the solution has a pH of about pH 5.5 to about pH 8.5. In embodiments, the solution has a pH of about pH 6 to about pH 8. In embodiments, the solution has a pH of about pH 6.5 to about pH 7.5. In embodiments, the solution has a pH of about pH 7. In one embodiment, the solution is a buffer. In some embodiments, the solution is a physiologic or cell-compatible (i.e., a solution in which a cell may survive for at least a short period of time) buffer or solution. In one embodiment, the buffer is phosphate-based. In one embodiment, the solution is a cell culture medium. The composition of the solution may be adjusted based on many factors and considerations, including, but not limited to, the frequency of the electromagnetic field used, the cell type used, or the foreign substance to be delivered into the cell. Osmolality and conductivity, as well as the presence or absence of ions such as potassium and calcium, may be adjusted as determined by a person skilled in the art.

In embodiments, the plurality of cells is substantially purified before and/or after exposing the plurality of cells to the electromagnetic field and before contacting the plurality of cells with the foreign substance. Many ways of substantially purifying cells are known to a person skilled in the art. As a non-limiting example, the cells may be pelleted by centrifugation and resuspended in a different solution. A person skilled in the art is knowledgeable as to how long and at which speed and temperature to centrifuge the cells at, depending on the specific cell type. Cells may also be purified by exploiting cellular surface markers or surface properties of the cells. As a non-limiting example, the cells may be substantially purified by incubating the cells with magnetic beads to bind to a target molecule on the cells, capturing the cells with a magnet, and replacing the solution surrounding the cells.

In embodiments, the plurality of cells is incubated with the foreign substance for at least about 10 s, about 20 s, about 30 s, about 40 s, about 50 s, about 60 s, about 90 s, about 120 s, about 150 s, about 180 s after exposure with HF EME. In embodiments, the plurality of cells is incubated with the foreign substance for at least about 4 min, at least about 5 min, at least about 6 min, at least about 7 min, at least about 8 min, at least about 9 min, or at least about 10 min. Incubation can be longer if needed, depending in the cell type.

After incubation of the cells with the foreign substance, the cells may or may not be substantially purified again. In embodiments, the cells are cultivated under conditions suitable for the given cell type.

### Foreign Substances

The methods disclosed herein allow for the delivery of a variety of different foreign substances to a cell or population of cells.

In one embodiment, the foreign substance is (or is conjugated to) a small molecule. The foreign substance may be (or may be conjugated to) a dye or a fluorescent molecule. In embodiments, the foreign substance is (or is conjugated to) cyanine, fluorescein, a fluorescein derivative (including, but not limited to fluorescein isothiocyanate), rhodamine, a rhodamine derivative (including, but not limited to tetramethyl rhodamine isothiocyanate (TRITC) or NHS-Rhodamine), or a maleimide activated fluorophore (including, but not limited to fluorescein-5-maleimide).

In one embodiment, the foreign substance is (or is conjugated to) a particle. In one embodiment, the particle is a nanoparticle, including, but not limited to, a nanosphere. The nanosphere may be a silica nanosphere. The particle may be fluorescent and/or magnetic.

In one embodiment, the foreign substance is (or is conjugated to) a nucleic acid. In embodiments, the nucleic acid is a deoxyribonucleic acid (DNA) or ribonucleic acid (RNA). In embodiments, the DNA is complementary DNA (cDNA), recombinant DNA, or genomic DNA. The DNA may be single or double-stranded. In some embodiments, the RNA is messenger RNA (mRNA), small RNA (sRNA), small interfering RNA (siRNA), small activating RNAs (saRNA), double-stranded RNA (dsRNA), micro-RNA (miRNA), long non-coding RNA (IncRNA), transfer RNA (tRNA), or small hairpin RNA (shRNA). The RNA may be single or double-stranded. The nucleic acid may comprise one or more modified nucleotides that increase the stability or half-life of the nucleic acid in vivo or in vitro. In one embodiment, the nucleic acid is methylated DNA. In one embodiment, the nucleic acid is a chromosome or a portion thereof. In embodiments, the nucleic acid is an expression vector or a plasmid. In embodiments, the nucleic acid encodes for a therapeutic polypeptide, a reporter protein, or a selectable marker (including, but not limited to a polypeptide conferring resistance to antibiotics). Non-limiting examples of reporter proteins are green fluorescent protein (GFP), red fluorescent protein (RFP), aquaporin, beta-galactosidase, uroporphyrinogen (urogen) III methyltransferase (UMT), and luciferase. Non-limiting examples of selectable markers include Blasticidin, G418/Geneticin, Hygromycin B, Puromycin, Zeocin, Adenine Phosphoribosyltransferase, and thymidine kinase.

The term “vector” means any molecule or entity (e.g., nucleic acid, plasmid, bacteriophage or virus) used to transfer protein or RNA coding information into a host cell. In some embodiments, a “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

In some embodiments, the foreign substance is (or is conjugated to) a polypeptide, a lipid, or a sugar. In one embodiment, the foreign substance is (or is conjugated to) a polysaccharide. In one embodiment, the foreign substance is (or is conjugated to) dextran.

In embodiments, the foreign substance is isolated or purified before used in the methods disclosed herein. As used herein, an “isolated” or “purified” foreign substance is substantially free of, for example, other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity can be measured by any appropriate standard method, for example, by column chromatography, thin layer chromatography, or high-performance liquid chromatography (HPLC) analysis. Isolated nucleic acid molecules according to the present disclosure further include molecules produced synthetically, as well as any nucleic acids that have been altered chemically and/or that have modified backbones.

Successful delivery of the foreign substance can be measured using methods known in the art. As a non-limiting example, if a nucleic acid comprising a transgene encoding a polypeptide is delivered to a cell, expression of the transgene may, for example, be determined by measuring the presence of mRNA (e.g., by RT-PCR) or by detecting the presence of the expressed polypeptide (e.g., by Western Blot, ELISA, flow cytometry, or, if applicable, by measuring enzymatic activity of fluorescence of the expressed protein).

### Cells

The methods disclosed herein are useful for the delivery of foreign substances to a variety of different cell types.

In one embodiment, the cell is a prokaryotic cell. Non-limiting examples of prokaryotic cells include bacterial cells (e.g., gram-positive, gram-negative, pathogenic, non-pathogenic, commensal, cocci, bacillus, and/or spiral-shaped bacterial cells) and archaea cells. In one embodiment, the cell is a bacterial cell. In one embodiment, the cell is a E. coli cell. In one embodiment, the cell is a JM109 E. coli cell. In some embodiments, the cell is a cell provided in Table 2.

In one embodiment, the cell is a eukaryotic cell. Non-limiting examples of eukaryotic cells include protozoan, algal, fungi, yeast, plant, animal, vertebrate, invertebrate, arthropod, mammalian, rodent, primate, and human cells. The cell may be a cell, e.g., of a unicellular organism or a multicellular organism. In one embodiment, the cell is a mammalian cell. In embodiments, the mammal is a mouse, dog, cat, horse, rat, goat, monkey, or rabbit. In one embodiment, the cell is a human cell. In one embodiment, the cell is a non-human mammalian cell. The cell may be an isolated cell. In embodiments, the cell is an embryonic stem cell, an induced pluripotent cell (iPSC), a primary cell, or a hematopoietic stem cell. In embodiments, the cell is an immune cell or an immune cell-derived cell, including, but not limited to, a T lymphocyte, a B lymphocyte, a dendritic cell, an antigen-presenting cell, a macrophage, a natural killer (NK) cell, a mast cell, a monocyte, a basophil, a eosinophil, or a neutrophil. In one embodiment, the cell is a cancer cell. In one embodiment, the cell is blood mononuclear cell. In one embodiment, the cell is a Jurkat cell. In one embodiment, the cells is a PC-12 cell. In one embodiment, the cells is a HeLa cell. In some embodiments, the cell is a chicken, frog, insect, or nematode cell.

In embodiments, provided is a plurality of cells that comprises a mixture of two or more different cell types. The plurality of cells may be a co-culture of multiple cell types (such as two or more of those disclosed herein) or a mixture of cell types that naturally occur together, such as in whole blood.

In embodiments, the plurality of cells comprises about 10 cells, about 10² cells, about 10³ cells, about 10⁴ cells, about 10⁵ cells, about 10⁶ cells, about 10⁷ cells, about 10⁸ cells, about 10⁹ cells, about 10¹⁰ cells, about 10¹¹ cells, or about 10¹² cells. In embodiments, the plurality of cells comprises about 10⁸ cells to about 10⁹ cells. In embodiments, the plurality of cells/ml comprises about 10 cells/ml, about 10² cells/ml, about 10³ cells/ml, about 10⁴ cells/ml, about 10⁵ cells/ml, about 10⁶ cells/ml, about 10⁷ cells/ml, about 10⁸ cells/ml, about 10⁹ cells/ml, about 10¹⁰ cells/ml, about 10¹¹ cells/ml, or about 10¹² cells/ml. In one embodiment, plurality of cells comprises about 8×10⁸ cells. In one embodiment, plurality of cells comprises about 8×10⁸ cells/mL.

The cells disclosed herein may be isolated from an animal or may have been cultivated in vitro for several days, weeks, or years. The cell may be derived from an immortalized cell line.

In embodiments, the cells into which the foreign substance is introduced are substantially isolated before being subject to the methods disclosed herein. In embodiments, a population of cells is substantially enriched for a specific cell type before being subject to the methods disclosed herein. Methods of isolating and enriching cells are known in the art. For example, cells may be enriched by virtue of their expression of cell surface markers or other identifying characteristics. As a non-limiting example, dendritic cells can be identified and isolated by virtue of their expression of the β-integrin, CD11c, or other identifying cell surface markers. With regard to cells, the term “isolated” means that the cell is substantially free of other cell types or cellular material with which it naturally occurs. For example, a sample of cells of a particular tissue type or phenotype is “substantially pure” when it is at least 60% of the cell population. Preferably, the preparation is at least 75%, more preferably at least 90%, and most preferably at least 99% or 100%, of the cell population. Purity is measured by any appropriate standard method, for example, by fluorescence-activated cell sorting (FACS).

Also provided is a cell that is produced according to any of the methods disclosed herein. The cell may have an altered cell property as a result of being exposed to a method disclosed wherein. The cell may be genetically modified.

### Applications of the Methods Disclosed Herein

The methods provided herein are useful for a variety of research and clinical applications. Numerous non-limiting examples are provided below.

Provided herein are methods for delivering polypeptides (including labeled polypeptides) to cells, allowing a person skilled in the art to study the polypeptide's half-life in cells, protein localization, or protein-protein interactions. Also provided are methods for delivering drugs into cells to screen for protein activity in the cytosol and to help identify protein therapeutics or to understand disease mechanisms. Provided is a method for delivering sugars into a cell to improve cryopreservation of the cell. Provided is a method for delivering nano particles or quantum dots into a cell for diagnostics and/or mechanic studies as well as introduction of quantum dots.

Provided is a method for delivering genetic or protein material to a cell to induce cell reprogramming to produce iPS cells. Provided is a method for delivering DNA and/or recombination enzymes into embryonic stem cells for the development of transgenic stem cell lines.

Provided herein is a method of stimulating antigen presentation in a cell by delivering an antigen to the cell. Provided herein is a method of isolating an immune cell from a patient, transfecting the immune cell with a nucleic acid using a method disclosed herein, and reintroducing the genetically modified immune cell into the patient. Provided herein is a method of isolating a dendritic cell from a patient, delivering to the dendritic cell an antigen using a method disclosed herein, and reintroducing the dendritic cell into the patient. For example, by delivering cancer antigens directly to the cytoplasm of the dendritic cell, one can exploit the MHC-I antigen presentation pathway and induce a powerful cytotoxic T lymphocyte (CTL) response in the patient. These activated T-cells then seek out and destroy any cancerous cells which express the target antigen. In embodiments, the delivered antigen is a commonly expressed protein known to be associated with a particular disease or a patient-specific one obtained from a biopsy.

Provided herein is a method of delivering a nucleic acid encoding a chimeric antigen receptor (CAR) to a T lymphocyte. Provided herein is a method of isolating T lymphocytes from a patient, transfecting the T lymphocytes with a nucleic acid encoding a CAR using a method disclosed herein, and reintroducing the genetically modified T lymphocytes into the patient.

Provided herein are methods of labeling cells for screening, imaging, or diagnostic purposes, by delivering into the cells a detectable marker, including, but not limited, to a fluorescent molecule, a radionuclide, a quantum dot, a gold nanoparticle, or a magnetic bead.

In embodiments, the method disclosed herein are used for nanoparticle mediated gene therapy and drug delivery.

It is to be understood that this disclosure is not limited to the particular molecules, compositions, methodologies, or protocols described, as these may vary. Any methods and materials similar or equivalent to those described herein can be used in the practice or testing of embodiments of the present disclosure. It is further to be understood that the disclosure of the invention in this specification includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments of the disclosure, and in the disclosure generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Transformation of Bacterial Cells Using HF EME

### Methods

E. coli Growth Conditions

E. coli JM 109 was purchased from Promega (Alexandria, NSW, Australia). Pure culture was stored at −80° C. in Luria-Bertani broth (LB) (Oxoid) supplemented with 20% (v/v) glycerol. The bacteria were thawed from −80° C. and were revived on LB agar (Oxoid) for 24 h at 37° C., and then stored at 4° C. For each independent experiment, fresh bacterial suspensions were prepared by inoculating a single colony in 100 mL LB and incubating overnight. Subsequently, the cell density was adjusted to 10⁸ CFU per mL (optical density at 600 nm [OD₆₀₀]=1.0) in 10 mM phosphate buffered saline (PBS; pH 7.4), using a spectrophotometer (Genesys 20 Visible Spectro, ThermoFisher). Of the prepared bacterial stock suspension, 2 mL of working suspension were transferred into a micro-Petri dish (35 mm diameter, Griener Bio One, Frickenhausen, Germany) for subsequent experiments.

Preparation of pGlo Plasmid DNA

The pGLO plasmid was purchased from BioRad (Gladesville, NSW, Australia) and extracted using the Wizard Plus SV Minipreps DNA Purification Systems according to the manufacturer protocol Promega (Alexandria, NSW, Australia). The plasmid encodes for a green fluorescent protein (GFP). As such, successful transformation can be detected by measuring GFP fluorescence.

To prepare a pGLO stock, E. coli JM 109 transformed with pGLO were grown overnight in LB broth supplemented with 100 mg/mL ampicillin at 37° C. 10 mL of the culture was centrifuged at 4900 rpm for 5 min and the cells resuspended in 250 μL of cell resuspension solution. 250 μL of the cell lysis solution was added and samples were inverted four times. Following that, 10 μL of alkaline protease solution was added and incubated for 5 min. After the addition of 350 μL neutralization solution added, the cells were centrifuged at 14,000 rpm for 10 mins leading to the production of the cleared lysate. The cleared lysate was collected into spin column and spun at 14,000 rpm for 1 min to allow binding of plasmid DNA. Finally, 750 μL of the wash solution was added and the plasmid DNA was collected to nuclease free water in the elution step. DNA concentration was recorded using the NanoDrop™2000/2000c Spectrophotometers (ThermoFisher) and samples were stored at −20° C.

Exposure of E. coli Cells to HF EME

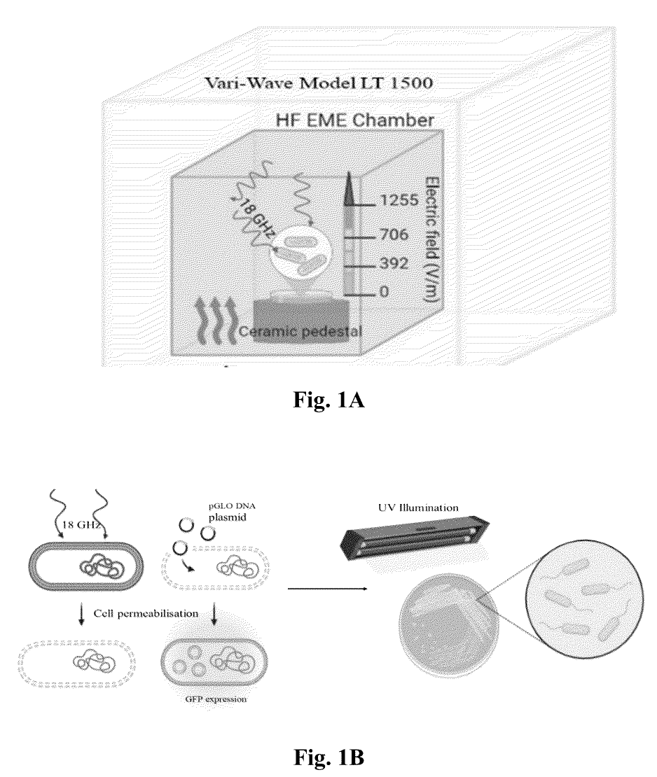

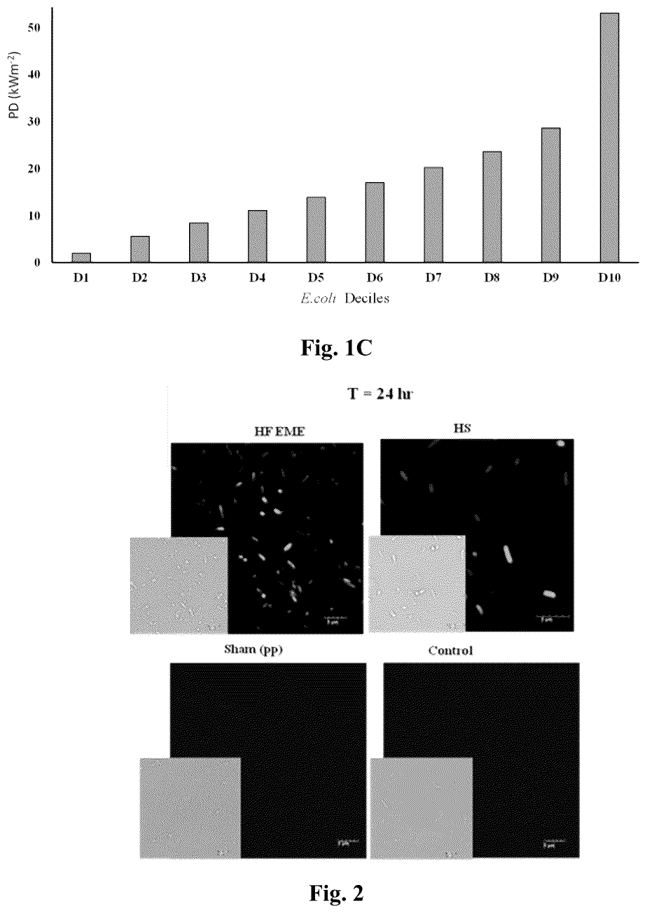

For the exposure of bacterial suspensions to 18 GHz EME, a Vari-Wave Model LT 1500 microwave (Lambda Technologies) was used that allowed for a varying frequency output range from 5-18 GHz (FIG. 1A). The sample was placed in the chamber on a ceramic pedestal (Pacific Ceramics, Sunnyvale, CA, US) in the hotspot free location which had been determined previously using CST Microwave Studio 3D Electromagnetic Simulation Software (CST of America, Framingham, MA, USA). The general experimental setup is shown in FIG. 1B. E. coli cells were resuspended in 2 mL of PBS (OD₆₀₀=1) and exposed to HF EME radiation at 18 GHz for 60 s, three times with a cooling period of 2 min in between exposures. The 2 min rest periods allowed for a reduction in the sample temperature and enabled the bacteria in the sample to change depth before undergoing the next exposure. The chamber was cooled to 25° C. during the 2 min resting period. The rise in temperature of the bacterial cell suspension was recorded for the first 60 s as described in Shamis, Y., et al., Review of the specific effects of microwave radiation on bacterial cells. Applied Microbiology and Biotechnology, 2012. 96: p. 319-325. The chamber had its core temperature monitored through the attachment of a fiber optic probe, temperature readings were taken every 15 s.

For the sham treatment control (Peltier treated control), the temperature profile during the HF EME exposures of 18 GHz was replicated using the Peltier plate (pp) heating/cooling system (TA Instruments, New Castle, DE, USA). An aliquot of 2 mL of the bacterial sample was loaded on the Peltier stage and was subjected to convection heating for 60 s, which was followed by cooling to 25° C. for 2 min before the application of the next heat treatment in order to replicate the changes in temperature profile experienced by HF EME treated cells. The temperature rise and fall was detected using a portable infrared/thermal monitoring camera (Cyclope 330S; Minolta, Osaka, Japan). The sham treated bacterial suspensions were analyzed following the same experimental protocols that were applied to HF EME exposed bacterial suspensions.

A portion of 2 mL of cells that had been treated with 18 GHz EME or sham treated controls were pelleted at 8000 rpm for 2 min, then resuspended in 500 μL of CaCl₂). This resuspended portion was then divided into 0.2 mL Eppendorf tubes at 50 μL aliquots. The pGLO plasmid at 100 ng/μL concentration was added and the cells incubated for 30 min on ice to allow for the uptake of the plasmid. Once the 30 min incubation period passed, the samples were transferred into separate 1.5 mL tubes and overgrown in 1 mL of LB broth in a shaker at 37° C. for 1 h. Once this was completed, the samples were plated on LB based agar plate mediums supplemented with 100 mg/u L ampicillin.

Heat Shock Transformation of E. coli Cells (Control Experiment)

The samples for heat shock were pelleted down at 8000 rpm for 5 min. The pellet was then resuspended in 0.5 mL of CaCl₂). From this suspension, a 50 μL aliquot was taken and 100 ng/u L of pGLO plasmid was added and incubated for 30 min on ice to allow for the uptake of the plasmid. After this period, the heat shock experiment was performed by lowering the sample into a 42° C. water bath for 30 s, after which they were chilled on ice for 5 min. The sample was then transferred in a similar fashion as the above sections into LB broth and shaken for 1 h at 37° C. and plated into LB plates supplemented with ampicillin.

### Confirmation of Bacterial Transformation

The transformation efficiency of E. coli cells was estimated using direct plate counting technique. A portion of 100 μL from serial dilutions were plated on LB plates containing 100 μg/μL ampicillin. Experimental groups exposed to heat shock, Sham treated and the untreated controls were prepared in the same manner.

Bacterial transformation efficiency was calculated as follows: Transformation efficiency=number of colony forming units: DNA (μg) spread on the plate.

Visualization of GFP in Transformed E. coli

Cells were outgrown in LB broth for 2 h and resuspended in 500 μL of PBS, aliquots of 50 μL were added onto 35 mm microscopy dishes (Ibidi® cells in focus, DKSH Australia Pty, Ltd. Mulgrave, Vic, Australia) for imaging using a Fluoview FV10i-W inverted microscope (Olympus, Tokyo, Japan). Fluorescence emission of GFP was measured by excitation at 395 nm and emission at 510 nm, different fields of view were captured as the bacterial cells were expressing the green signal.

To construct the total fluorescence (CTF) plots, CLSM was used to capture ten different fields of view of each sample, the CTF was calculated using ImageJ software (Image J plugin; National Institute of Health, Bethesda, MD, USA) by manually selecting an area across the image using the Frechand ROI tool. The parameters were set selecting the area, integrated density, and mean grey value. After analyzing the fluorescence intensity, a smaller region of the image that has no fluorescence was selected as the background and taken into account when constructing the plots.

### Flow Cytometry (FC)

FC calibration was conducted using a reference bead mix, the FC tubing was washed in 10% bleach to remove if any particles might have had remained within the tubing. The wash was conducted at a sample flow rate of 15 L/min, which was then followed by another was of 0.22 μm sterile filtered PBS to remove the bleach. Bleach and PBS washes were conducted before a sample change of the different experimental samples. Serial dilutions of bacterial suspensions were prepared in 0.22 μm sterile filtered PBS. The measurements were adjusted to a flow rate of 25 μL/min, stop condition: 5000 events. The voltage was adjusted accordingly and the fcs files were generated using the Invitrogen Attune NxT Flow Cytometer (ThermoFisher Scientific). The resulting fcs files were processed with the Floreada analysis tool available at https://floreada.io (accessed on 11 Nov. 2022). In order to exclude debris, which displayed low FSC values, from the analyses, gating in the FSC vs. SSC plot was performed for every determination.

### Transmission Electron Microscopy (TEM)

Cells were resuspend in CaCl₂) and briefly spun at 1000 rpm for 30 s and resuspended in LB broth. Bacterial suspensions were applied onto carbon foam wire grids at a volume of 10 μL and incubated for 2 min. The excess solution was removed by tilting the grid and gently tapping onto a filter paper. The grid was then washed in sterile ultrapure H₂O for 30 s and stained with 1% uranyl acetate for 30 s and the final wash conducted in sterile ultrapure H₂O for 60 s. The excess solution was dried by dragging the grid on filter paper, the grids were air dried for overnight before imaging using the JEM 1010 instrument (JEOL). Approximately 20 TEM images were taken at 5000× for sample analysis.

### Statistical Analysis

Statistical data processing was conducted using the Statistical Package for the Social Sciences, SPSS 24.0 (SPSS, Chicago, IL, USA). The homogeneity of the variances was tested using Levene's test and statistically significant differences (p<0.05, p<0.01) among the various groups were calculated using a one-way ANOVA analysis with the independent variables in the study being the four different conditions of treatment. Five independent technical replicates were performed for each sample group, each sample group had 3-5 replicates.

### Results

Estimation of EME Energy Delivered to E. coli Cells

E. coli cells, in a 2 mL suspension with physiological buffer, were subjected to HF EME delivered at 18 GHz for a total of 180 s. Following every discrete 60 s exposure, the sample volume and chamber were cooled to 25° C., and the cells were mixed. The amount of energy delivered (as incident power density, PD) to the bacterial cells over the three exposures, specific absorption rate (SAR) of the cells, and instantaneous change in temperature ΔT was calculated as described below.

The amount of energy I_{d} that is delivered to a sample by an incident beam at a given depth can be calculated using the equation I_{d}=Iₒe^{-dα} (equation 1). Iₒ is the incident intensity, d is the depth, e is Euler's number and α is the absorption coefficient.

E. coli cells have an estimated water content of 74%. As such, the amount of incident EME can be calculated as for water. Biological molecules such as lipids, carbohydrates, and proteins have a much lower α at GHz frequencies when compared to water, in the order of 1 cm⁻¹ or less, and do not undergo a profound change in their properties. Estimating the intensity of the incident EME within a sample at GHz frequencies is complex since the dielectric properties of water undergo a rapid frequency and temperature dependent change in the 1-100 GHz range. For instance, the α of water at 20° C. is approximately 19.3 cm⁻¹ at 18 GHZ, and is reduced to 14.9 cm⁻¹ at 40° C. Given the range of the absorption coefficient (a) of water between 14.9 cm⁻¹ (at 40° C.) to 19.3 cm⁻¹ (at 20° C.) at 18 GHz within the experimental temperature range, the intensity of the radiation from the top to the bottom of the sample varied as follows: approximately 1.8% of the radiation reached the bottom of the sample at 25° C. and 4.5% at 40° C. A median a of 17 cm⁻¹ was used for the calculation of the exposure intensities.

Minimizing the sample depth reduces the EME absorption differences within the sample. The practical depth, however, is limited by the need for a manageable sample size (2 ml), minimizing evaporation, and having an even PD.

The PD was calculated according to the EME required to raise the sample temperature by 15° C. (from 25° C. to 40° C.). Given that the volume of a typical E. coli bacterium is approximately 1.5 μm³, the concentration of the bacteria by volume in the solution was in the order of 0.015%. The contribution of the non-water components of the cells could be safely neglected in calculating the total energy needed.

Using the heat capacity of water of 4182 J/kg K, and equation Q=c(mΔT) (equation 2), where Q is the total energy, c is the heat capacity for water, and m is the mass of the sample, the energy required to raise 2 mL (2 g) of water 15° C. during each 60 s exposure was calculated to be 125.5 Joules (if the heat loss during the 60 s exposure itself was ignored). This required an input of 2.09 Js⁻¹ (i.e., Watts) to the sample, over an area of (0.000962 m⁻²), giving an incident PD of 2173 Wm⁻² to the surface of the sample.

The loss of heat to the surrounding environment during the exposure itself is, however, sizable and also needs to be considered. It can be approximated using Newton's law of cooling, which can be expressed as dT/dt=k(Ti−Ta) (equation 3), where dT is the change in temperature, dt the change in time, k is a constant, whilst Ti and Ta are the initial sample and ambient temperatures, respectively. Given that the sample required 120 s to cool from 40° C. to 25° C., using an ambient temperature of 0° C. (placed on ice), according to Newton's law of cooling, the constant k was calculated to be in the order of 0.031. Using the derived value for k, and using the exponential form of Newton's law of cooling dT=e^{-kt} (equation 4), the heat loss during the exposure was approximately equivalent to the energy required to raise the sample by 6.4° C. The additional energy required for this can be estimated by using equation 2, and the specific heat of water. It was in the order of 27.0 J for each heating episode. The PD required for this over 60 s was approximately 467 Wm⁻². When added to the estimated PD required to elevate the sample temperature by 15° C. of 2173 Wm⁻², the total incident PD was calculated to be approximately 2640 Wm⁻². When considering the total exposure of 180 s, the total energy absorbed in the sample was ˜460 J.

The root mean square (RMS) value of the incident electric field can be calculated by using the equation PD=Eₒ×Hₒ (equation 5), wherein Eₒ is the incident RMS electric field strength, and Hₒ is the incident RMS magnetic field strength. In air, where impedance of free space Z₀=376.73Ω, giving the strength of H of approximately

$$\frac{1}{377}$$

of E, the equation 5 can then be approximated by Eₒ=sqrt (PD×377) (equation 6). This gives an incident RMS Eₒ value of 997 Vm⁻¹. For PD within the sample, the refractive index (n) of the sample must also be included in the calculations. Since the magnetic permeability of water is within 0.0008% of that of free space, the value for PD within the sample can be approximated by: PD=n E_{D}×H=n E_{D}²/377 (equation 7), where E_{D} is the RMS electric field strength at depth D and Eₒ is the incident RMS electric field strength. Similar to α, n of water also undergoes rapid, temperature dependent, change at GHz frequencies. At 18 GHz, n of water is 6.7 at 25° C. and 7.3 at 40° C. α is related to E_{D} using the equation E_{D}=sqrt(Eₒ²e^{-αD}) (equation 8). Since the bacteria undergo both active motile and Brownian motion in suspension, the dispersion of the exposure profiles of the single bacteria will vary considerably.

E. coli cells following exposure to 18 GHz EME had a calculated mean power density (PD) of 15.2 kWm⁻², and the median was calculated to be 14.0 kWm⁻². The calculated percentiles of the averaged PD received by the E. coli population over the 3 exposures are shown in FIG. 1C.

The specific absorption rates (SAR) in the sample vary considerably. The equation for SAR can be written as SAR=σE²/ρ (equation 9), where σ is the frequency specific (AC) conductivity of the material, and ρ is the mass density. The σ of water at 18 GHz is also temperature dependent, with approximate values of 35 Sm⁻¹ at 20° C. and 30 Sm⁻¹ at 40° C. The σ and ρ of E. coli was assumed to be 20 Sm⁻¹ and 1100 kg m⁻³, approximated by using the dielectric parameters of tissue with similar hydration, protein, and lipid composition.

The initial temperature rise ΔT in the E. coli cells can then be calculated by using the equation (ICNIRP): ΔT=(SAR/cₜ) Δt, where cₜ is the heat capacity of the tissue. The value for E. coli was approximated to other biological tissues, and set at 3400 J/kg K.

The calculated PD, SAR and ΔT are given in Table 1 for sample depths at 0.2 m depth intervals. The PD and rise in temperature decrease as depth increases within the irradiated sample.

TABLE 1
Incident power density calculated at various depths of sample.

| Depth | PD | SAR | Temperature rise |
|---|---|---|---|
| (mm) | (kWm⁻²) | (kW kg⁻¹) | (° C./s) |
| 2.0 | 18.5 | 18 | 5.3 |
| 1.8 | 13.0 | 13 | 3.7 |
| 1.6 | 9.2 | 9.0 | 2.6 |
| 1.4 | 6.5 | 6.3 | 1.9 |
| 1.2 | 4.6 | 4.5 | 1.3 |
| 1.0 | 3.2 | 3.1 | 0.92 |
| 0.8 | 2.3 | 2.2 | 0.65 |
| 0.6 | 1.6 | 1.6 | 0.46 |
| 0.4 | 1.1 | 1.1 | 0.32 |
| 0.2 | 0.79 | 0.8 | 0.23 |
| 0.0 | 0.56 | 0.5 | 0.16 |

Uptake of the pGLO Plasmid by E. coli Cells

The efficiency of bacterial transformation with HF EME was compared to transformation using heats hock. Sham treated cells (exposure to conventional heating mimicking the 3 discrete 60 s temperature profile rises using a Peltier plate system, and in the absence of HF EME) were used as a control. After HF EME exposure, Peltier plate heating (sham), or heat shock treatment, respectively, the bacteria were resuspended in calcium chloride (CaCl₂)) to promote the binding of plasmid DNA to lipopolysaccharides. Expression was GFP was assessed using fluorescence microscopy, flow cytometry, and direct plate counting.

As shown in FIGS. 2-4, an incident EME PD between 560 and 18400 Wm⁻² is sufficient to induce genetic transformation in E. coli cells with pGLO plasmid DNA following exposure to the HF EME of 18 GHZ.

First, the uptake of pGLO was confirmed using confocal microscopy by quantifying fluorescence intensity of expressed GFP (FIG. 2). 24 h post exposure, the number of cells expressing GFP in the HF EME treated sample was 402±19.4 per mm². The number of cells expressing GFP in the heat shock group was 327±6.2 per mm² and the ‘no DNA’ control group and the sham group exhibited no fluorescence.

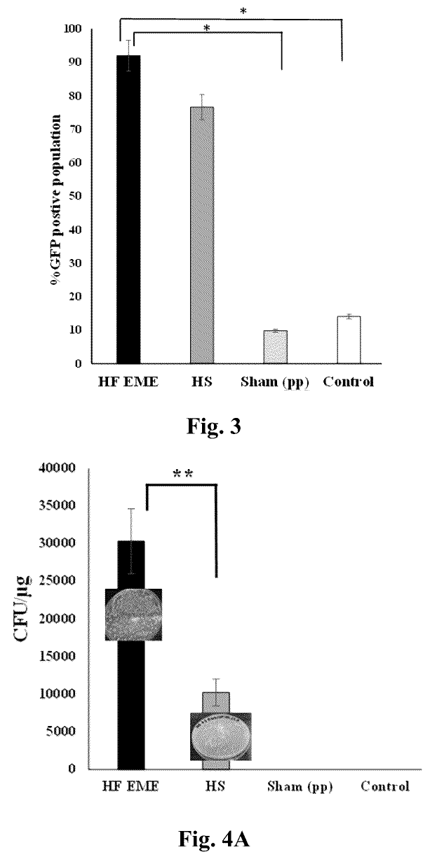

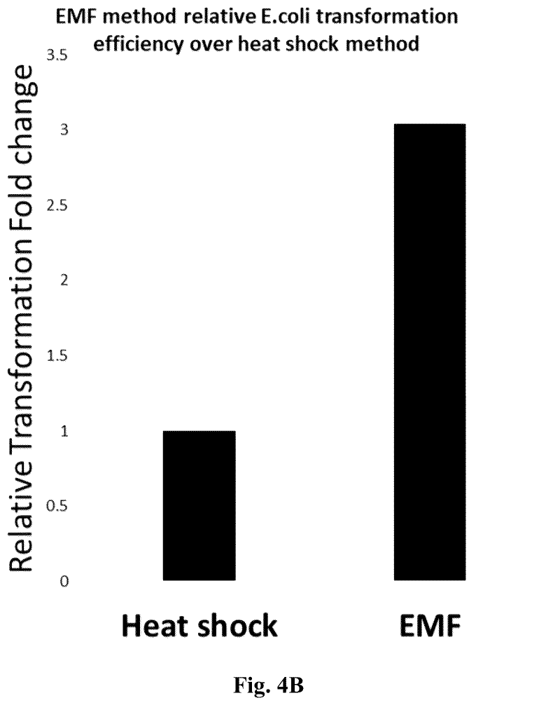

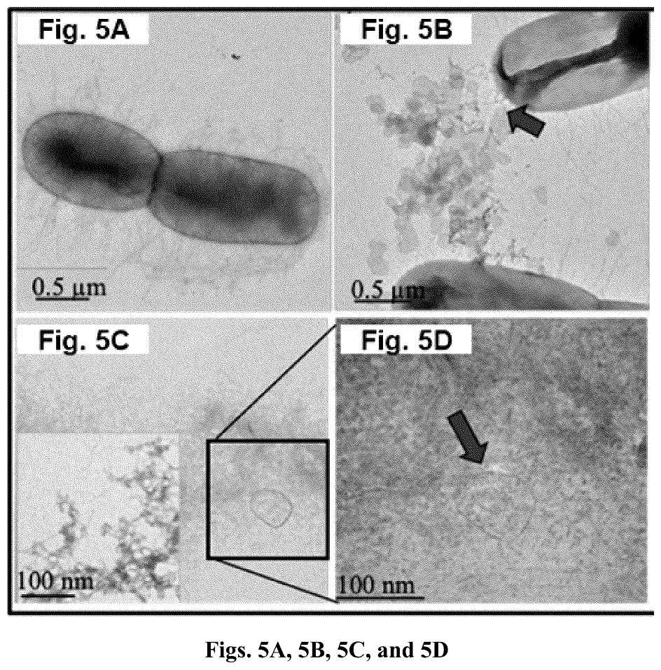

Next, the successful transformation E. coli cells with pGLO plasmid DNA was analyzed using flow cytometry. The percentage of GFP expressing cells in the entire cell population was quantified (FIG. 3). The HF EME-exposed group displayed 90.73±1.9% GFP-positive cells, whereas the HS-treated group exhibited 76.91±5.4% GFP-positive cells. A 90.73% uptake suggests that the threshold for the effect of the 18 GHz PD was in the order of 3.5 kW m⁻² or less, under the current experimental set up. Positive GFP peaks were not detected for the Sham (pp) exposed and the control groups, given the E. coli cells population, and there were no GFP expressing colonies observed on the agar plates following culture of E. coli on growth selective medium.

Finally, transformation efficiency was assessed using direct plate counting. The pGLO plasmid contains two origins of replication and several sites for restriction endonucleases within the plasmid genome. The pGLO plasmid also carries a gene for ampicillin resistance which allows the differentiation between transformants and the non-transformed cells, since the growth of cells not carrying the pGLO plasmid will be inhibited by the ampicillin in the growth medium. Transformation led to a significantly higher number of colony forming units (CFU) as compared to the heat shock technique (FIG. 4).

In sum, HF EME induced genetic transformation in E. coli cells was calculated to have a transformation efficiency of 3.0×10⁴ CFU/μg of the pGLO plasmid DNA, which is three time greater in comparison to the transformation efficiency of heat shock transformation of E. coli cells (1.0×10⁴ CFU/μg of pGLO plasmid DNA). A comparison of the transformation efficiency achieved by HF EME with known transformation efficiencies is shown in Table 2.

TABLE 2
Transformation efficiencies of different methods of bacterial transformation.
Heat shock and electroporation are the standard methods used in genetic
transformation of bacteria yielding efficiencies the range of 10⁴-10⁹ CFU/μg.

| Transformation | | | Efficiency | |
|---|---|---|---|---|
| method | E. coli strains | Plasmid | (CFU/μg DNA) | Reference |
| HF EME | JM109 | pGLO | 9.4 × 10⁴ | This study |
| Heat shock | C600 | R-factor DNA | 10⁴ | Ref. 1 |
| Heat shock | C600 | pSC101 | 3 × 10⁵ | Ref. 2 |
| Heat shock | C600 | R6-5 | 2.8 × 10⁴ | Ref. 2 |
| Heat shock | C600 | Mixture of pSC101 | 2 × 10⁵ | Ref. 2 |
| | | and pSC102 | | |
| Heat shock | χ1776 | pBR322 | >10⁷ | Ref. 3 |
| Heat shock | C600 | NTP16 | 5 × 10⁸ | Ref. 4 |
| Heat shock | JM109 | pUC19 | 1 × 10⁶ | Ref. 5 |
| Heat shock | HB101, C600, | pBR322 | 5 × 10⁷ to 1 × 10⁹ | Ref. 6 |
| | XL1-blue, | | | |
| | JM105 and | | | |
| | JM109 | | | |
| Heat shock | DH5α | pGLO | 1 × 10⁶ | Ref. 7 |
| Electroporation | LE392 and | pUC18 and | 10⁹ to 10¹⁰ | Ref. 8 |
| | DH5α | pBR329 | | |
| Electroporation | K803 and | pB1221.23 and | 10⁸ to 10⁹ | Ref. 9 |
| | DH10B | pBSK+ | | |
| Electroporation | XL1-blue | pBluescriptIISK(+) | 10⁸ | Ref. 10 |
| Electroporation | DH5α | BAC plasmid | 7 × 10⁸ | Ref. 11 |
| Electroporation | DH5α, TOP10 | pUC19 and pGEM- | >10⁷ | Ref. 12 |
| | and JM109 | T easy | | |

### REFERENCES CITED IN TABLE 2

Ref 1: Cohen, S. N., A. C. Chang, and L. Hsu, Nonchromosomal antibiotic resistance in bacteria: genetic transformation of Escherichia coli by R-factor DNA. Proceedings of the National Academy of Sciences, 1972. 69 (8): p. 2110-2114.
Ref 2: Cohen, S. N., et al., Construction of biologically functional bacterial plasmids in vitro. Proceedings of the National Academy of Sciences, 1973. 70 (11): p. 3240-3244.
Ref 3: Norgard, M. V., K. Keem, and J. J. Monahan, Factors affecting the transformation of Escherichia coli strain χ1776 by pBR322 plasmid DNA. Gene, 1978. 3 (4): p. 279-292.
Ref 4: Weston, A., et al., Transformation of Escherichia coli with plasmid deoxyribonucleic acid: calcium-induced binding of deoxyribonucleic acid to whole cells and to isolated membrane fractions. Journal of bacteriology, 1981. 145 (2): p. 780-787.
Ref 5: Chung, C., S. L. Niemela, and R. H. Miller, One-step preparation of competent Escherichia coli: transformation and storage of bacterial cells in the same solution. Proceedings of the National Academy of Sciences, 1989. 86 (7): p. 2172-2175.
Ref 6: Tang, X., et al., The optimization of preparations of competent cells for transformation of E. coli. Nucleic acids research, 1994. 22 (14): p. 2857.
Ref 7: Sha, J., et al., Heat-shock transformation of Escherichia coli in nanolitre droplets formed in a capillary-composited microfluidic device. Analytical Methods, 2011. 3 (9): p. 1988-1994.
Ref 8: Dower, W. J., J. F. Miller, and C. W. Ragsdale, High efficiency transformation of E. coli by high voltage electroporation. Nucleic acids research, 1988. 16 (13): p. 6127-6145.
Ref 9: Sawahel, W., et al., Development of an electro-transformation system for Escherichia coli DH10B. Biotechnology Techniques, 1993. 7 (4): p. 261-266.
Ref 10: Chuang, S. E., A. L. Chen, and C. C. Chao, Growth of E. coli at low temperature dramatically increases the transformation frequency by electroporation. Nucleic Acids Res, 1995. 23 (9): p. 1641.
Ref 11: Nováková, J., et al., Improved method d for high-efficiency electrotransformation of Escherichia coli with the large BAC plasmids. Folia Microbiologica, 2014. 59 (1): p. 53-61.
Ref 12: Liu, J., et al., An Improved Method of Preparing High Efficiency Transformation Escherichia coli with Both Plasmids and Larger DNA Fragments. Indian Journal of Microbiology, 2018. 58 (4): p. 448-456.
   Interactions of the pGLO Plasmid with E. coli Cells

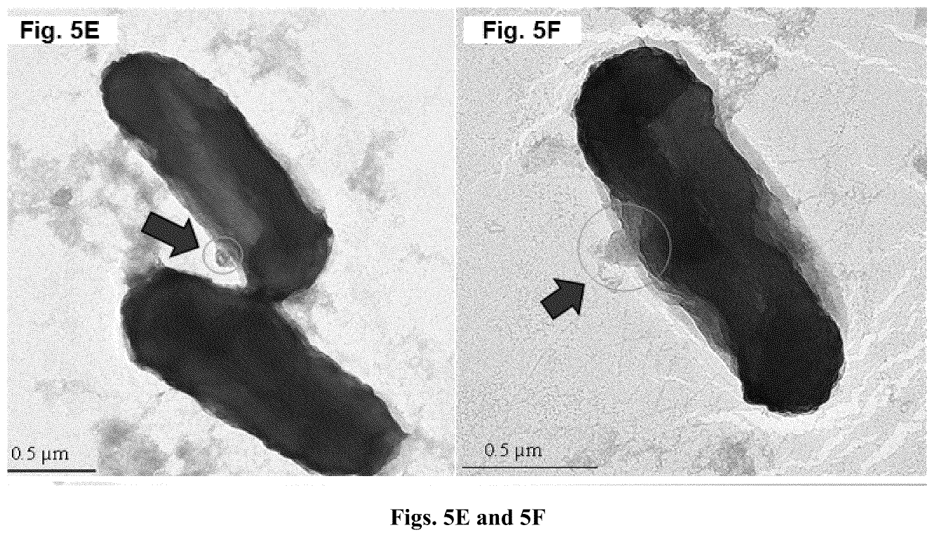

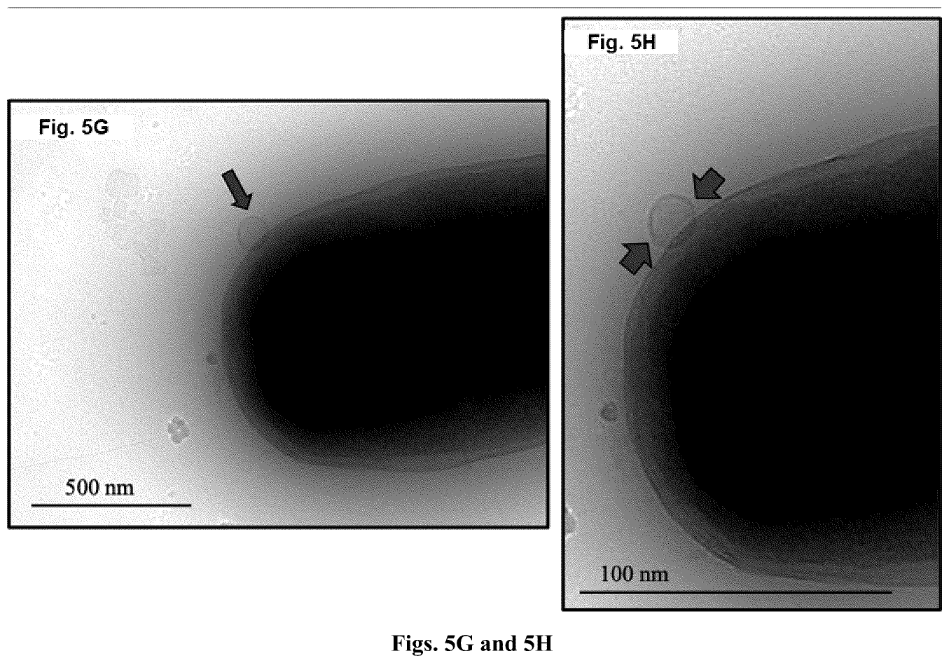

Interactions of plasmid DNA and E. coli cells exposed to HF EME were examined using TEM. The pGLO plasmid used for bacterial transformation has a length of 344.3 nm in its double helical structure. Following extraction of the pGLO plasmid, restriction enzyme EcoRI was used to digest the plasmid to obtain the linear form and to confirm the presence of pGLO. The plasmid DNA in its extracted form was used for transformation. TEM micrographs revealed that the pGLO plasmid DNA interacts with the bacterial cell (FIGS. 5E and 5F, arrows point towards the bacterial membrane, where the DNA is been internalized following HF EME exposure). In contrast, in unexposed E. coli cells, no internalization of plasmid DNA or interaction of DNA with the cell membrane could be observed.

In sum, provided herein is a technique applicable for the genetic transformation of bacterial cells without compromising cell viability. HF EME treated cells remained viable. Given the high a of water at GHz frequencies, the pGLO uptake by E. coli cells over a wide PD dispersion indicates that the HF EME induced genetic transformation can be applied at a large diversity of sample depths and in a range of physiological temperatures. This makes the HF EME technique versatile and robust beyond what can be produced by other means.

### Example 2: Transfection of Jurkat Cells Using HF EME

### Methods

### Cells Origin and Growth Conditions

The Jurkat cell line used in this study was Clone E6-1 (ATCC® TIB-152™) purchased from the American Type Culture Collection (Manassas, VA, USA). It is a human T lymphoblastoid cell line derived from an acute T cell leukemia. Cells were cultured in a complete Gibco™ RPMI 1640 medium (ThermoFisher Scientific, Waltham, MA, USA) supplemented with 10% Gibco™ fetal bovine serum (ThermoFisher Scientific), 10 mM Gibco™ HEPES buffer solution (ThermoFisher Scientific), and 1% Gibco™ penicillin/streptomycin (ThermoFisher Scientific). Supplements were stored as aliquots at −20° C. Stock solutions of the Jurkat cells were prepared in a medium containing 20% FBS, 10 mM HEPES, 1% penicillin/streptomycin, and 5% dimethyl sulfoxide and stored in liquid nitrogen. The cells were maintained at 37° C. with 5% CO₂ in a 95% humidified incubator. The medium was changed every 2 days and passaged accordingly when the confluence reached 90%.

Jurkat cells were activated using ImmunoCult™ Human CD3/CD28/CD2 T Cell Activator (STEMCELL Technologies, Tullamarine, VIC, Australia). Briefly, approximately 5 million viable Jurkat cells in fresh growth medium at 5×10⁵ cells/mL were added with 25 μL/mL of ImmunoCult™ Human CD3/CD28/CD2 T Cell Activator and incubated at 37° C. and 5% CO₂ for 3 days before each experiment.

### Exposure of Cells to HF EME

For EME exposure, a Vari-Wave Model LT 1500 microwave was used (see Example 1). The EME apparatus (Vari-Wave Model LT 1500; Lambda Technologies, Morrisville, NC, USA) used in the study has an option of varying the frequency range from 5 to 18 GHz. The frequency was adjusted to a fixed value of 18 GHz and the power ranged from 17 W. Before exposure to EME, the cell density for the Jurkat cells was adjusted to 1×10⁶ cells/mL in Opti-MEM I reduced-serum medium (ThermoFisher Scientific) using a hemocytometer (Paul Marienfeld GmbH & Co KG, Lauda-Konigshofen, Germany). A micro petri dish (35 mm diameter; Griener Bio-One, Frickenhausen, Germany) with the sample (2 mL) was placed on the ceramic pedestal (Pacific Ceramics, Sunnyvale, CA, USA; ε'=160, loss tangent<10⁻³) on the hot spot-free location, identified by electric field modelling using CST Microwave Studio 3D Electromagnetic Simulation Software (CST of America, Framingham, MA, USA) and experimental temperature measurements. The temperature rise in the cell suspension was monitored using a built-in temperature probe, a Luxtron Fiber Optic Temperature Unit (LumaSense Technologies, Santa Clara, CA, USA). The surface area that was exposed to the EME was approximately 9.62 cm², giving a sample depth of 2.08 mm. The cells were subjected to three discrete 45-second continuous exposure cycles with a cooling period of 2 min in between the exposures. The EME chamber was cooled during the 2 min resting period using ice packs to bring the temperature to 25° C. The 2 min rest periods allowed for a reduction of the sample temperature and allowed the cells in the sample to mix and change depth before undergoing the next exposure. The temperature was maintained below 37° C.

### Cellular Uptake of Silica Nanospheres

Fluorescent silica nanospheres with a diameter of 23.5±0.2 nm (fluorescein isothiocyanate [FITC]) (Corpuscular Inc, Cold Spring, NY, USA) were used to study the permeability of both resting and activated Jurkat cells. Immediately following EME exposure, the nanospheres were added into the cell suspension at a concentration of 10 μg/mL. After 10 minutes of incubation, the samples were washed twice using PBS and centrifugation at 1,300 rpm for 5 minutes at 25° C. The same procedure was repeated for the unexposed control. A 150 μL aliquot of the sample was visualized using a Fluoview FV10i-W inverted microscope (Olympus Corporation, Tokyo, Japan). The samples were analyzed immediately but not incubated for 24 h and 48 h to avoid the passive and natural uptake of nanospheres inside the cells.

### Transmission Electron Microscopy (TEM)

After the nanospheres incubation and washing steps following EME exposure, cell suspensions were pelleted by centrifugation at 1,300 rpm for 5 minutes at 25° C. The cell pellet was then resuspended in primary fixative of 2% paraformaldehyde and 2.5% glutaraldehyde in 0.1 M sodium cacodylate buffer (pH 7.4) overnight at 4° C. The next day, cells were washed thrice in cacodylate buffer for 10 minutes each. Then, cells were postfixed in 1% osmium tetroxide and 1.5% paraformaldehyde in distilled water for 1 hour, followed by three washes in distilled water for 10 minutes each and left overnight at 4° C. After that, cells were dehydrated through a graded ethanol series (50%, 70%, and 90%) for 15 minutes and then further dehydrated by passing through 100% ethanol series twice followed by 100% acetone series for 30 minutes at 25° C. Then, cells were further infiltrated with a 1:1 ratio of acetone:Spurr's resin mixture overnight. After that, cells were completely exchanged in 100% Spurr's resin twice for 3 hours each. The resin samples were further polymerized at 70° C. for 48 h. The final block was trimmed, then cut into ultrathin sections (90 nm thickness) using a Leica Ultracut Ultramicrotome (Leica Microsystems, Wetzlar, Germany) with a diamond knife (Diatome, Hatfield, PA, USA). Sections were placed onto 200 mesh copper grids and examined using a JEM 1010 instrument (JEOL, Tokyo, Japan). Approximately 40 TEM images were taken at ×5,000 and ×10,000 magnifications for sample analysis.

mRNA Transfection

The DasherGFP mRNA (Aldevron, Fargo, ND, USA) was added to the EME exposed and unexposed samples in naked form and in lipid complexes form by using Lipofectamine 2000 (ThermoFisher Scientific). The mRNA encodes a fluorescent protein optimized for expression in mammalian cells. In order to deactivate any RNAase contaminations in the experiment, Ambion™ RNAsecure™ (ThermoFisher Scientific) was diluted to 1× final concentration in the Opti-MEM I reduced-serum medium. The mixture was then incubated at 60° C. for 10 minutes and then cooled down to 37° C. This mixture was then used to adjust the Jurkat cell's density and to prepare the mRNA-Lipofectamine complexes for transfection. For the naked mRNA transfection, 2 mL of the EME exposed and unexposed Jurkat cell suspensions were added each with 100 pmol of mRNA directly and incubated for 10 minutes before transferring to a T25 culture flask. The mixture was topped up with 2 mL of fresh growth medium and incubated at 37° C. and 5% CO₂.

mRNA-Lipofectamine complexes were employed as a positive control and to assess the enhancement of EME on transfection efficiency. The complexes were prepared as recommended by the manufacturer. Briefly, 100 pmol of mRNA were diluted gently with 250 μL of the mixture Opti-MEM I reduced-serum medium with RNAsecure. Then, 5 μL of Lipofectamine were also diluted gently with 250 μL of the mixture Opti-MEM I reduced-serum medium with RNAsecure and incubated for 5 minutes at 25° C. After that, the diluted mRNA and diluted Lipofectamine were combined gently to form complexes and further incubated for 20 minutes at 25° C. The complexes were then added each to 2 mL of the EME exposed and unexposed Jurkat cell suspensions and incubated for 10 minutes before transferring to a T25 culture flask. The mixture was topped up with 2 mL of fresh growth medium and incubated at 37° C. and 5% CO₂. The transfection efficiency of the transfected cells was assessed and compared with the unexposed cells immediately after transfection, 24 h, and 48 h after incubation.

### Cell Viability

The viability of Jurkat cells was determined using the LIVE/DEAD Viability/Cytotoxicity Kit (ThermoFisher Scientific). The viability of the EME exposed cells was monitored and compared with the unexposed control at 0 hour, 24 h, and 48 h post treatment through three individual replicates. CLSM was used in assessing the number of viable cells; 10 fields of view were analyzed per sample type.

### Flow Cytometry

Transfection efficiency was evaluated by flow cytometry in terms of Green Fluorescent Protein (GFP) expression in the cells 24 h after exposing cells to 18 GHz EME. The flow cytometer Invitrogen Attune Nxt (ThermoFisher Scientific) was set to collect data on 100,000 cells with excitation at 488 nm and detection of green fluorescence. The resulting fcs files were processed with the Floreada analysis tool available at https://floreada.io (accessed on 7 Mar. 2023). The same number of cells was evaluated for each sample, correcting for any difference in cell density.

### Statistical Analysis

Statistical data processing was conducted using the Statistical Package for the Social Sciences, SPSS 24.0 (SPSS, Chicago, IL, USA). The statistically significant differences (p<0.05) among the various groups were calculated using Tukey-Kramer procedure for one-way ANOVA analysis. Three independent technical replicates were performed for each sample group, each sample group had three replicates.

### Results

### HF EME-Induced Uptake of Nanospheres

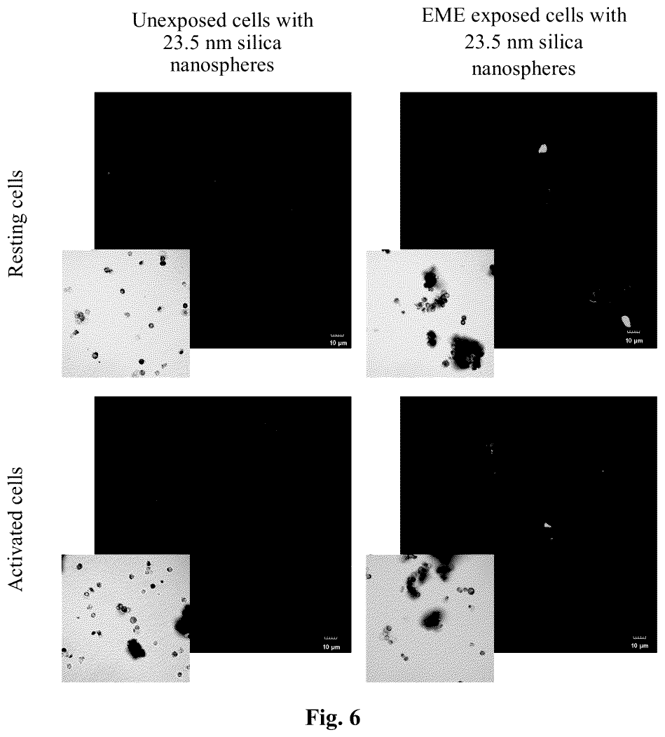

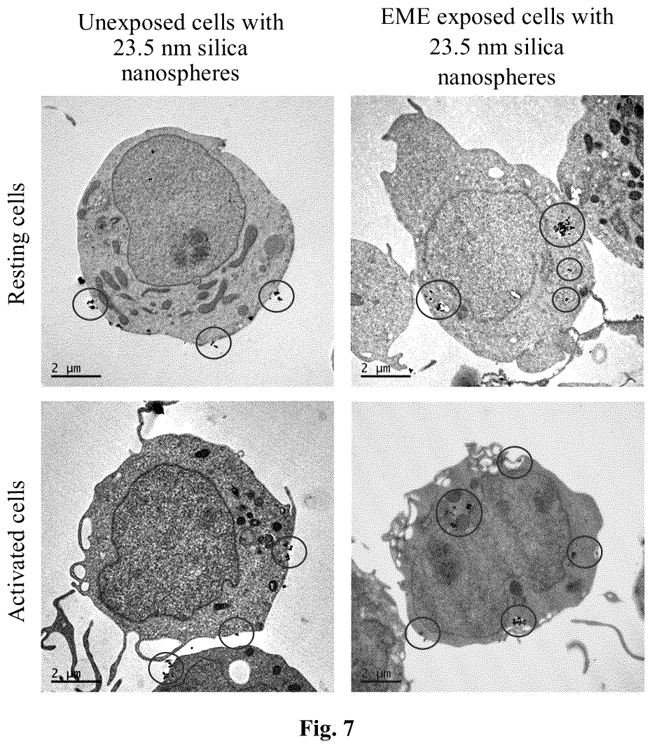

The 18 GHz EME triggered a transient increase in membrane permeability in both resting and activated Jurkat cells, as confirmed by the rapid internalization of silica nanospheres (d=23.5 nm), which is evident in CLSM and TEM images (FIGS. 6 and 7, respectively). In the CLSM analysis (FIG. 6), there were a smaller number of resting and activated unexposed control cells also had a fluorescent signal from the silica nanospheres. This could be due to the natural ability of nanospheres of this size to passively penetrate inside the cells and/or the nanospheres could be stuck on the cell membranes. As such, TEM analysis was performed to confirm the location of silica nanospheres in the cells. TEM images showed that the nanospheres (circles) were located inside the cytoplasm of both resting and activated Jurkat cells after 18 GHz EME exposures. No internalized nanospheres were detected in TEM images for the unexposed control group. Some nanosphere clusters could be seen sticking on the cell membranes.

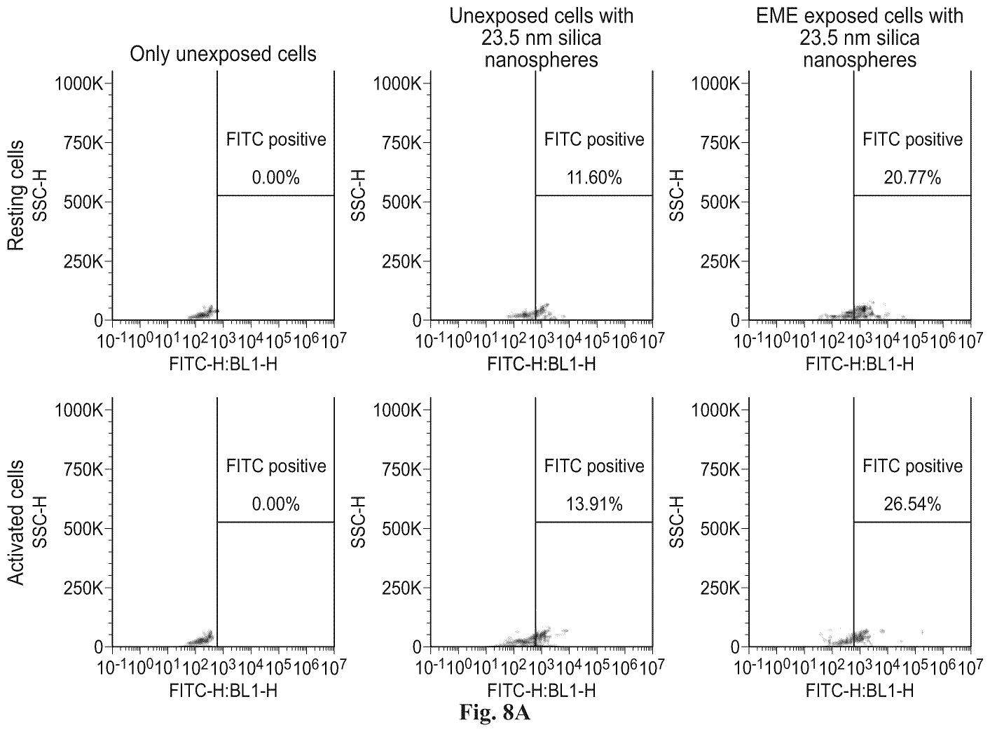

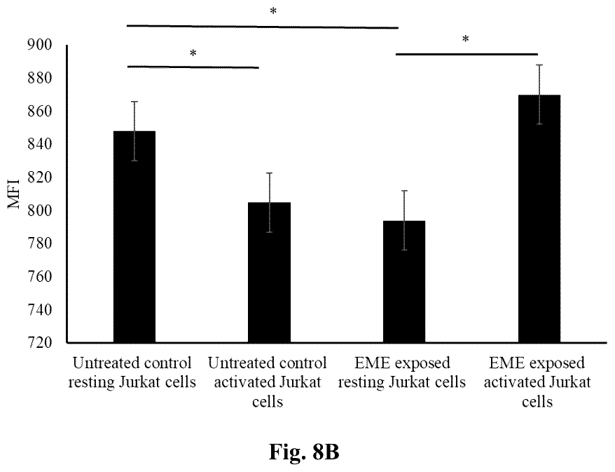

The proportion of nanosphere-internalized cells in all experimental groups was further investigated using flow cytometry. About 13.9% of the activated Jurkat cells internalized the silica nanospheres. There was a 2.3% increasement in the number of activated cells internalized nanospheres naturally in comparison to the resting untreated cells (FIG. 8A). The amount of activated Jurkat cells which had the nanospheres inside rose almost double (approximately 12.6%) from 13.9% to 26.5% of the whole population after exposed to 18 GHz EME. The Median Fluorescence Intensity (MFI) values correlated very well with the percentage of fluorescent cells suggesting a good signal to noise ratio and there were actual shifts in the fluorescent intensity of the samples. There were statistically significant differences for the silica uptake level between EME exposed and unexposed in both the resting and activated Jurkat cells (FIG. 8B).

HF EME-Induced of Jurkat Cells with mRNA

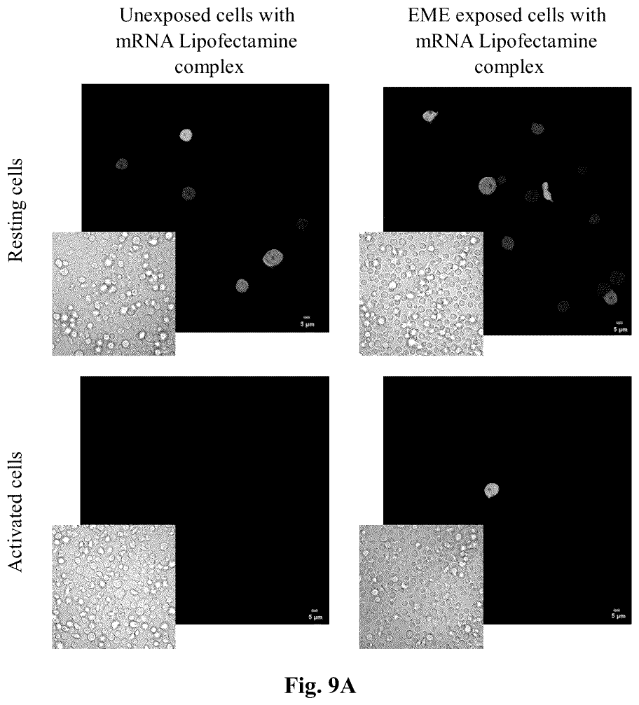

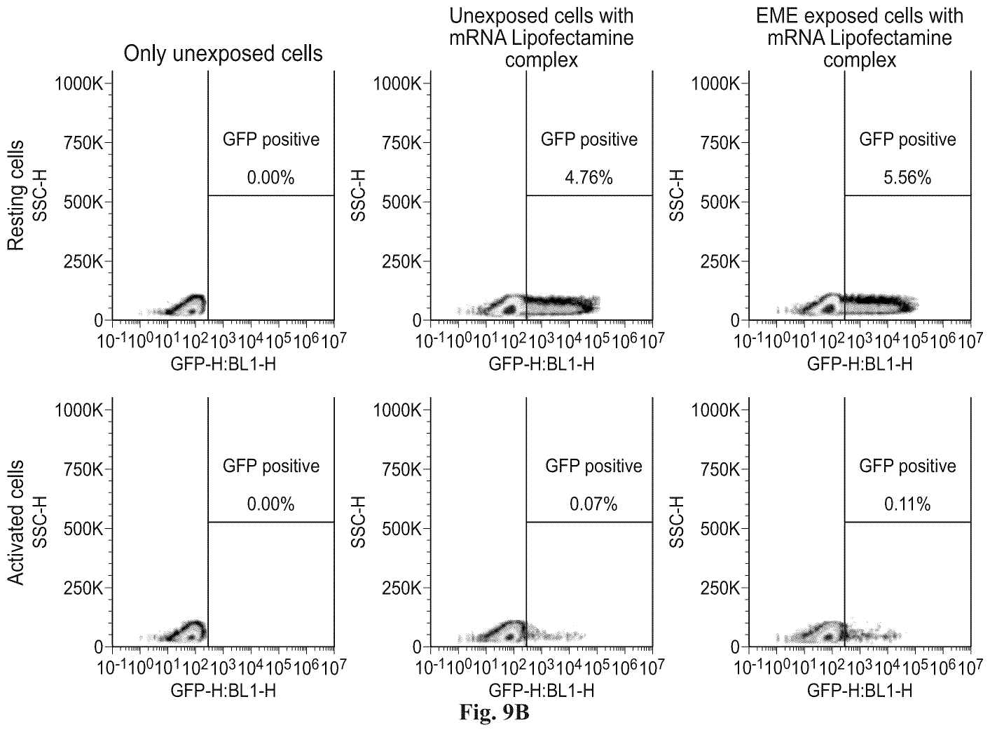

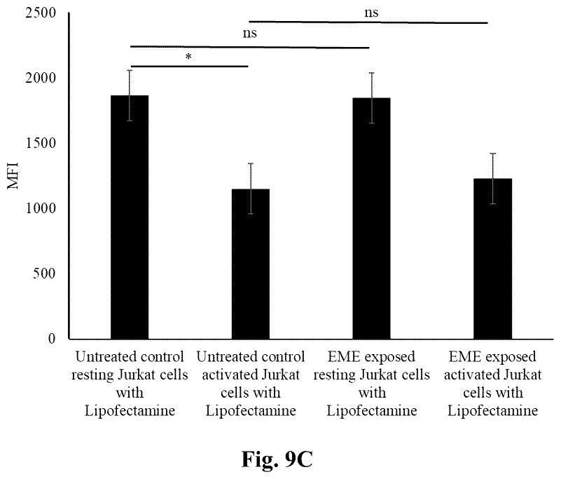

After exposing to 18 GHz EME, resting and activated Jurkat cells were successfully transfected with mRNA-lipofectamine complex and stably expressed GFP after 24 h of incubation. Transfection efficiency of the different experimental samples was analyzed using independent complementary techniques such as CLSM and flow cytometry (FIG. 9A).

The proportion of successfully mRNA transfected cells in all experimental groups was further investigated using flow cytometry. There was approximately a 4.69% decrease in the number of activated cells expressed GFP in comparison to the resting unexposed control cells (FIG. 9B). The amount of activated Jurkat cells which expressed GFP rose approximately 0.04% from 0.07% to 0.11% of the whole population after exposing to 18 GHz EME. The MFI values correlated very well with the proportion of fluorescent cells suggesting a good signal to noise ratio and there were actual shifts in the fluorescent intensity of the samples. There was no statistically significant difference for the GFP expression level between EME exposed and unexposed in both the resting and activated Jurkat cells (FIG. 9C).

### Cell Viability of 18 EME Exposed Jurkat Cells

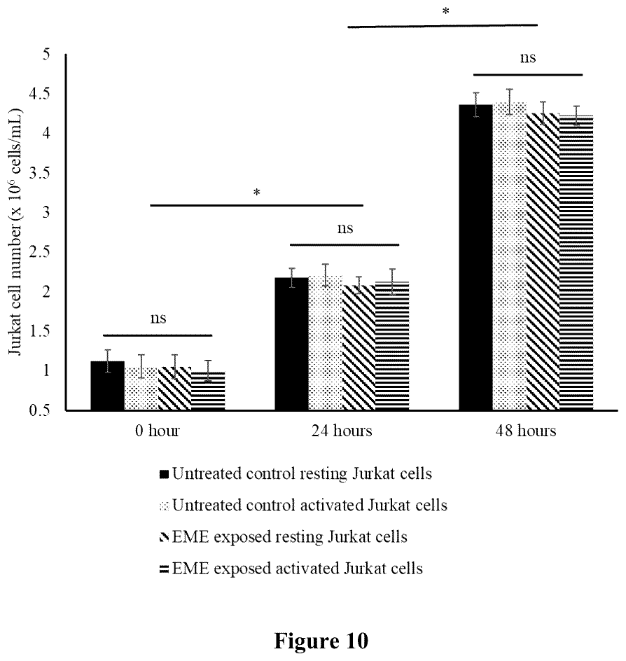

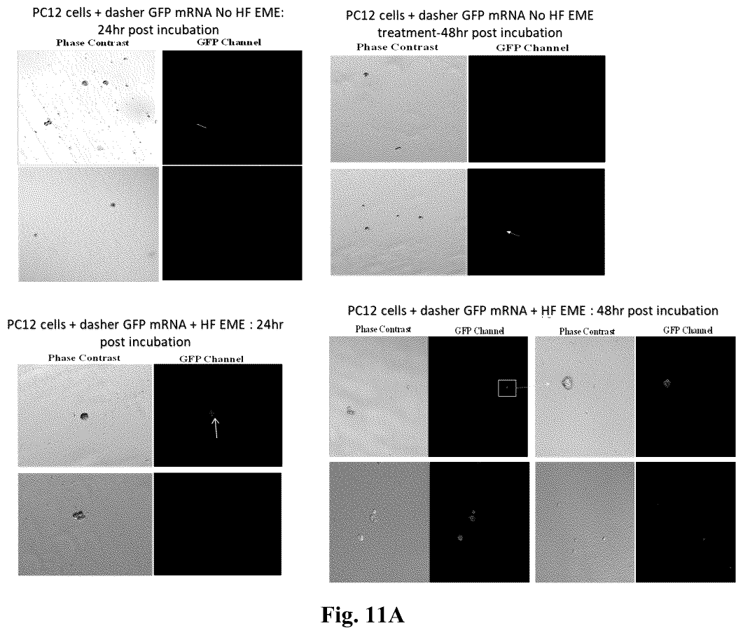

The viability of Jurkat cells after 18 GHz EME exposure was investigated using CLSM (FIG. 10). Visual examination of the fluorescence micrographs showed that the cells remained highly viable (>93%), indicating that the 18 GHz EME exposure did not affect cell viability. A statistical analysis of the data did not reveal a statistically significant difference between the viability of the EME-exposed and the unexposed control cells (P>0.05). Cells in all the samples were able to grow and double after 24 h and 48 h. There were statistically significant differences for the viable cell number between 0 hour, 24 h, and 48 h of growth time in all the samples. This finding has an important implications for the use of 18 GHz EME as an innovative physical method to achieve a temporary increase in membrane permeability for, e.g., drug or genetic material delivery, where cell death is undesirable.

In sum, the use of EME allowed for the successful update of 23.5 nm silica nanospheres as well as mRNA an a difficult-to-transfect mammalian cell model without any detrimental effect to cell viability. These cells are known to be resistant to common chemical transfection reagents, particularly with the lipofectamine 2000, which has an efficiency of about 2-3%.

### Example 3: Transfection of PC-12 Cells Using HF EME

### Methods

### Cells Origin and Growth Conditions

PC-12 is a cell line derived from a pheochromocytoma of the rat adrenal medulla. The PC-12 cell line used in this study was purchased from the American Type Culture Collection (ATCC, USA) and cultured in a complete Gibco™ RPMI medium (ThermoFisher Scientific, Australia) supplemented with 10% Gibco™ horse serum (ThermoFisher Scientific, Australia, HS), 5% Gibco™ fetal bovine serum (ThermoFisher Scientific) (FBS) and 1% Gibco™ penicillin/streptomycin (ThermoFisher Scientific) (PS). Supplements were stored as aliquots at −20° C. Stock solutions of the PC-12 cells were prepared in a medium containing 90% FBS and 10% DMSO and stored in liquid nitrogen. The cells were maintained at 37° C. with 5% CO2 in a 95% humidified incubator. The medium was changed every two days and passaged accordingly when the confluence reached 90%.

### EME (18 GHz) Treatment of PC-12 Cells

For the EME treatment of PC-12 cells, the cells were exposed to EME in 30 s-long cycles, which allowed for the temperature to be maintained below 37° C. (any increases in temperature were monitored). For the treatment, the frequency of a Lambda Technologies Vari-Wave Model LT 1500 was adjusted to a fixed frequency of 18 GHZ and the power ranged from 17 W. In brief, the micro petri dish (35 mm diameter, Griener Bio One, Frickenhausen, Germany) with the sample was placed on the ceramic pedestal (Pacific Ceramics, Sunnyvale, CA, USA, insert symbol, =160, loss tangent<10-3) on the hotspot free location, identified by electric field modelling using CST Microwave Studio 3D Electromagnetic Simulation Software (CST MWS) (CST of America, Framingham, MA, USA) and experimental temperature measurements. The cell density of PC-12 used for EME exposure was adjusted to 6×10⁴ cells/mL in phosphate buffered saline (PBS) using a hemocytometer (Paul Marienfeld GmbH&Co.KG Lauda-Konigshofen, Germany). The PC-12 cell suspensions were exposed to EME for a duration of 30 s. The temperature rise in the cell suspension was monitored using a built-in temperature probe, a LuxtronFiber Optic Temperature Unit (LFOTU) (LumaSense Technologies, Santa Clara, CA, USA). After the MW treatment, the sample was cooled down to 25° C. for 2 min. The microwave chamber was cooled using ice packs to bring the temperature to 25° C. The sample was exposed to three cycles (30 s; 2 min cooling) of MW radiation while keeping all the other environmental factors constant. Immediately following the exposures, the cells were pelleted at 1300 rpm for 4 min, resuspended in 50 μL of PBS and 500 ng/μL of the pEGFP-N1 plasmid or Dasher eGFP mRNA. The pEGFP-N1 plasmid is suitable for expression of GFP in mammalian cells. The plasmid also promotes bacterial resistance to kanamycin, allowing for propagation in E. coli cells. Dasher eGFP mRNA was purchased from Aldevron (Fargo, ND, USA). The samples were incubated for 5 min for genetic uptake. After the incubation, the samples were washed with 1×PBS and the PC-12 cells were resuspended in a complete Gibco™ RPMI medium (ThermoFisher Scientific, Australia) supplemented with 10% Gibco™ horse serum (ThermoFisher Scientific, Australia, HS), 5% Gibco™ FBS and 1% Gibco™ penicillin/streptomycin (ThermoFisher Scientific, Australia, PS) and seeded onto poly-L-lysine coated micro petri dishes. Cells were then incubated for 24 h or 48 h at 37° C. with 5% CO₂ in a 95% humidified incubator.

### Confocal Scanning Laser Microscopy Imaging of PC-12 Cells

CLSM was used in assessing the uptake by analyzing approximately 10 fields per sample. A 150 μL aliquot of each sample was visualized using a Fluoview FV10i-W inverted microscope (Olympus Corporation, Tokyo, Japan).

### Results

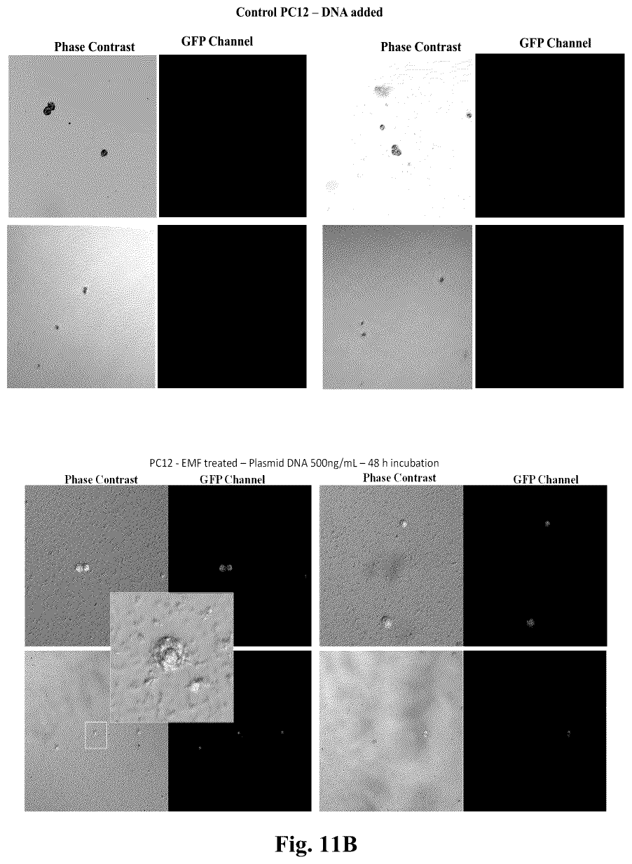

As shown in FIG. 11, EME was successfully used to transfect PC-12 cells with mRNA (FIG. 11A) as well as plasmid DNA (FIG. 11B), illustrating that the methods disclosed herein work with different eukaryotic cells.

We claim:

1. A method for introducing a foreign substance into a cell, the method comprising:
(a) providing a plurality of cells suspended in a first solution;
(b) exposing the plurality of cells to an electromagnetic field with a frequency of about 6 to about 35 GHz;
(c) allowing the temperature in the first solution to decrease;
(d) repeating steps (b) and (c) one or more times; and
(e) contacting the plurality of cells with the foreign substance,
wherein the temperature of the first solution in steps (b) to (d) does not exceed about 37° C.

2. The method of claim 1, wherein steps (b) and (c) are repeated two, three, four, five, or six times prior to step (e).

3. The method of claim 1, wherein steps (b) and (c) are repeated twice prior to step (e).

4. The method of claim 1, wherein the plurality of cells is exposed to the electromagnetic field for about 30 to about 120 s in step (b).

5. The method of claim 1, wherein in step (c) the temperature in the first solution is allowed to decrease for about 30 s to about 300 s.

6. The method of claim 5, wherein in step (c) the temperature in the first solution is allowed to decrease for about 60 s to about 200 s.

7. The method of claim 6, wherein in step (c) the temperature in the first solution is allowed to decrease for about 120 s.

8. The method of claim 1, wherein the plurality of cells is exposed to the electromagnetic field for about 45 s in step (b), the temperature in the first solution is allowed to decrease for about 120 s in step (c), and wherein steps (b) and (c) are repeated two times.

9. The method of claim 1, wherein the plurality of cells is exposed to the electromagnetic field for about 60 s in step (b), the temperature in the first solution is allowed to decrease for about 120 s in step (c), and wherein steps (b) and (c) are repeated two times.

10. The method of claim 1, wherein the plurality of cells is exposed to the electromagnetic field for about 30 s in step (b), the temperature in the first solution is allowed to decrease for about 120 s in step (c), and wherein steps (b) and (c) are repeated five times.

11. The method of claim 1, wherein in step (c) the temperature in the first solution is allowed to decrease to about 20° C., about 21° C., about 22° C., about 23° C., or about 24° C.

12. A method for introducing a foreign substance into a cell, the method comprising:
(a) providing a plurality of cells suspended in a first solution;
(b) exposing the plurality of cells to an electromagnetic field with a frequency of about 6 to about 35 GHz; and
(c) contacting the plurality of cells with the foreign substance,
wherein the temperature of the first solution in step (b) does not exceed about 37° C.

13. The method of claim 12, wherein the method is performed in a device comprising a temperature control unit.

14. The method of claim 12, wherein step (b) is repeated two, three, four, five, or six times prior to step (c).

15. The method of claim 12, wherein the plurality of cells is exposed to the electromagnetic field for about 30 to about 120 s in step (b).

16. The method of claim 1, wherein the electromagnetic field has a frequency of about 10 to about 30 GHz.

17. The method of claim 1, wherein the first solution is a first buffer.

18. The method of claim 1, wherein the first solution has a pH of about 6 to about 8.

19. The method of claim 1, wherein the foreign substance is a nucleic acid, polypeptide, sugar, or a small molecule.

20. The method of claim 19, wherein the nucleic acid is a deoxyribonucleic acid (DNA) or ribonucleic acid (RNA).

21. The method of claim 20, wherein the nucleic acid is a DNA or RNA comprising one or more modified nucleotides that increase the stability or half-life of the DNA or RNA in vivo or in vitro.

22. The method of claim 20, wherein the DNA is methylated DNA.

23. The method of method of claim 19, wherein the nucleic acid is a naturally occurring chromosome or a portion thereof.

24. The method of claim 19, wherein the nucleic acid is an expression vector.

25. The method of claim 19, wherein the nucleic acid comprises a transgene encoding for a polypeptide or an RNA.

26. The method of claim 25, the method further comprising a step of incubating the plurality of cells under conditions allowing for the expression of the polypeptide or RNA after the step of contacting the plurality of cells with the foreign substance.

27. The method of claim 25, wherein the RNA is a siRNA, antisense oligonucleotide, or an RNAi.

28. The method of claim 26, wherein the step of incubating the plurality of cells is performed at about 37° C.

29. The method of claim 1, wherein the plurality of cells is a plurality of bacterial cells or eukaryotic cells.

30. The method of claim 29, wherein the bacterial cells are Escherichia coli cells.

31. The method of claim 29, wherein the eukaryotic cells are immune cells, immune cell-derived cells, primary cells, pluripotent cells, or hematopoietic stem cells.
